SEQUENCE LISTING (27 sequences)

SEQ ID NO: 1 - DNA, 25 bp, Artificial Sequence
  Probe-1
  misc_feature 1: cytosine is modified with a 6-FAM
  misc_feature 25: thymine is modified with a IQ-4
catcattcacccttggcacaggtgt

SEQ ID NO: 2 - DNA, 25 bp, Artificial Sequence
  Probe-2
  misc_feature 1: cytosine is modified with a 6-FAM
  misc_feature 25: thymine is modified with a BHQ2
catcattcacccttggcacaggtgt

SEQ ID NO: 3 - DNA, 20 bp, Artificial Sequence
  Forward Primer
cccatgttcgtcatgggtgt

SEQ ID NO: 4 - DNA, 24 bp, Artificial Sequence
  N1 Probe
accccgcattacgtttggtggacc

SEQ ID NO: 5 - DNA, 23 bp, Artificial Sequence
  N2 Probe
acaatttgcccccagcgcttcag

SEQ ID NO: 6 - DNA, 24 bp, Artificial Sequence
  N3 Probe
aycacattggcacccgcaatcctg

SEQ ID NO: 7 - DNA, 20 bp, Artificial Sequence
  N1 Forward Primer
gaccccaaaatcagcgaaat

SEQ ID NO: 8 - DNA, 24 bp, Artificial Sequence
  N1 Reverse Primer
tctggttactgccagttgaatctg

SEQ ID NO: 9 - DNA, 20 bp, Artificial Sequence
  N2 Forward Primer
ttacaaacattggccgcaaa

SEQ ID NO: 10 - DNA, 18 bp, Artificial Sequence
  N2 Reverse Primer
gcgcgacattccgaagaa

SEQ ID NO: 11 - DNA, 22 bp, Artificial Sequence
  N3 Forward Primer
gggagccttgaatacaccaaaa

SEQ ID NO: 12 - DNA, 21 bp, Artificial Sequence
  N3 Reverse Primer
tgtagcacgattgcagcattg

SEQ ID NO: 13 - DNA, 22 bp, Artificial Sequence
  Reverse Primer
ggtcatgagtccttccacgata

SEQ ID NO: 14 - DNA, 25 bp, Artificial Sequence
  Probe-1
  misc_feature 1: cytosine is modified with a 6-FAM
  misc_feature 25: cytosine is modified with a IQ-4
ctgcaccaccaactgcttagcaccc

SEQ ID NO: 15 - DNA, 25 bp, Artificial Sequence
  Probe-2
  misc_feature 1: cytosine is modified with a 6-FAM
  misc_feature 25: cytosine is modified with a BHQ1
ctgcaccaccaactgcttagcaccc

SEQ ID NO: 16 - DNA, 25 bp, Artificial Sequence
  Probe-3
  misc_feature 1: cytosine is modified with a 6-FAM
  misc_feature 25: cytosine is modified with a BHQ2
ctgcaccaccaactgcttagcaccc

SEQ ID NO: 17 - DNA, 24 bp, Artificial Sequence
  Probe-1
  misc_feature 1: adenine is modified with a 6-FAM
  misc_feature 24: cytosine is modified with a IQ-4
accccgcattacgtttggtggacc

SEQ ID NO: 18 - DNA, 24 bp, Artificial Sequence
  Probe-2
  misc_feature 1: adenine is modified with a 6-FAM
  misc_feature 24: cytosine is modified with a BHQ1
accccgcattacgtttggtggacc

SEQ ID NO: 19 - DNA, 24 bp, Artificial Sequence
  Probe-3
  misc_feature 1: adenine is modified with a 6-FAM
  misc_feature 24: cytosine is modified with a BHQ2
accccgcattacgtttggtggacc

SEQ ID NO: 20 - DNA, 23 bp, Artificial Sequence
  Probe-1
  misc_feature 1: adenine is modified with a 6-FAM
  misc_feature 23: guanine is modified with a IQ-4
acaatttgcccccagcgcttcag

SEQ ID NO: 21 - DNA, 23 bp, Artificial Sequence
  Probe-2
  misc_feature 1: adenine is modified with a 6-FAM
  misc_feature 23: guanine is modified with a BHQ1
acaatttgcccccagcgcttcag

SEQ ID NO: 22 - DNA, 23 bp, Artificial Sequence
  Probe-3
  misc_feature 1: adenine is modified with a 6-FAM
  misc_feature 23: guanine is modified with a BHQ2
acaatttgcccccagcgcttcag

SEQ ID NO: 23 - DNA, 24 bp, Artificial Sequence
  Probe-1
  misc_feature 1: adenine is modified with a 6-FAM
  misc_feature 24: guanine is modified with a IQ-4
aycacattggcacccgcaatcctg

SEQ ID NO: 24 - DNA, 24 bp, Artificial Sequence
  Probe-2
  misc_feature 1: adenine is modified with a 6-FAM
  misc_feature 24: guanine is modified with a BHQ1
aycacattggcacccgcaatcctg

SEQ ID NO: 25 - DNA, 24 bp, Artificial Sequence
  Probe-3
  misc_feature 1: adenine is modified with a 6-FAM
  misc_feature 24: guanine is modified with a BHQ2
aycacattggcacccgcaatcctg

SEQ ID NO: 26 - DNA, 22 bp, Artificial Sequence
  Forward Primer
acagctgtgtggtgcttctgtg

SEQ ID NO: 27 - DNA, 22 bp, Artificial Sequence
  Reverse Primer
cattgtcctctgtccaggcatc

(19) United States
(12) Patent Application Publication
(10) Pub. No.: US 2021/0355551 A1
(43) Pub. Date: Nov. 18, 2021

(54) RT-qPCR Molecular Detection and Diagnosis of 2019 Novel Coronavirus

(71) Applicants: Suresh C. Srivastava, Wilmington, MA (US); ChemGenes Corporation, Wilmington, MA (US)

(72) Inventor: Suresh C. Srivastava, Burlington, MA (US)

(21) Appl. No.: 16/874,647

(22) Filed: May 14, 2020

(51) Int. Cl. C12Q 1/70

(52) U.S. Cl. CPC C12Q 1/70; C12Q 2600/16

(57) ABSTRACT

Provided herein are oligonucleotide probes for detecting 2019 novel coronavirus (2019-nCoV). The probes are modified at their 5′ ends with a fluorophore (e.g., fluorescein) and at their 3′ ends with a moiety capable of quenching fluorescence from the fluorophore. The moiety is based on the IQ-4 or IQ-2 quencher. Also provided are kits including one or more of such oligonucleotide probes, and methods of detecting 2019-nCoV and/or diagnosing COVID-19 using the oligonucleotide probes and kits described herein.

### INCORPORATION BY REFERENCE OF MATERIAL IN ASCII TEXT FILE

This application incorporates by reference the Sequence Listing contained in the following ASCII text file being submitted concurrently herewith:
a) File name: 58021001000SEQUENCELISTING.txt; created May 14, 2020, 7 KB in size.

### BACKGROUND

In late December, 2019, patients presenting with viral pneumonia due to an unidentified microbial agent were reported in Wuhan, China. A novel coronavirus was subsequently identified as the causative pathogen, provisionally named 2019 novel coronavirus (2019-nCoV). As of Jan. 26, 2020, more than 2,000 cases of 2019-nCoV infection had been confirmed, most of which involved people living in or visiting Wuhan, and human-to-human transmission had been confirmed. Lu, R., et al. Lancet 2020; 395:565-574.

Reverse transcription quantitative polymerase chain reaction (RT-qPCR) has been the industry standard in vitro diagnostic test to detect 2019-nCoV. Primers and hydrolysis probes targeting specific sequences in the viral RNA genome are the basis for the RT-qPCR technology, which reverse transcribes RNA into complementary DNA (cDNA), amplifies cDNA by PCR, and can ultimately be used to quantify the amount of RNA in the original sample using fluorescence. Each target-specific RT-qPCR probe comprises a sequence targeting a specific sequence in the viral RNA genome, a fluorophore attached to one end of the probe sequence and a quencher attached to the other end. Close proximity of the quencher to the fluorophore attenuates the fluorescence of the fluorophore. The amount of fluorescence at the end of each PCR cycle can be detected and registered by a qPCR instrument, and corresponds to the amount of cDNA and, therefore, RNA containing the targeted region. The quencher's ability to efficiently attenuate fluorescence from the fluorophore when physically linked and unmask fluorescence when cleaved minimizes background fluorescence and maximizes sensitivity, which is crucial in obtaining accurate RT-qPCR results.

The Centers for Disease Control and Prevention (CDC) have approved Black Hole Quencher (BHQ)-1 for use in the 2019-nCoV RT-qPCR Diagnostic Panel, and have published the recommended probes and primer sequences. BHQ-2 has been shown to perform at a greater efficiency than BHQ-1, and is one of the industry-leading quenchers available. However, due to an unprecedented demand in 2019-nCoV detection RT-qPCR Diagnostic Panels, the supplies of BHQ-1 and BHQ-2 have been strained, resulting in the lack of RT-qPCR Diagnostic Panels around the globe. Furthermore, the CDC 2019-nCoV RT-qPCR Diagnostic Panel targets just two viral genes—N1 and N2—due to the lower detection limit of N3, which is likely related to the sensitivity of the fluorophore and quencher pair. The lower detection limit of N3 gene has huge public health consequences: if a RT-qPCR test of N1 and N2 genes detects one gene but not the other, the test is deemed inconclusive. This uncertainty in infection can create fear or a false sense of health, and repeating the test further strains already limited resources.

Being able to detect N3, and include it in the Diagnostic Panel would increase the detection power of the CDC 2019-nCoV RT-qPCR Diagnostic Panel, and allow more certainty in declaring a test result positive or negative. Accordingly, there is a need for an oligonucleotide probe that can detect multiple fragments of 2019-nCoV.

### SUMMARY

This technology is based, at least in part, on the discovery that an oligonucleotide probe comprising a probe sequence modified with a dye pair including an IQ-4-based quencher (available from ChemGenes Corporation) can detect multiple fragments of the nucleocapsid (N) gene of a 2019-nCoV, including the N3 gene fragment of a 2019-nCoV.

Accordingly, provided herein is an oligonucleotide probe for detecting a 2019 novel coronavirus (2019-nCoV). The oligonucleotide probes comprises a probe sequence complementary to the complementary DNA (cDNA) sequence of the sequence of a N1, N2 or N3 gene of the 2019-nCoV, or a fragment thereof. The probe sequence is modified at its 5′ terminus with a fluorophore having an emission maximum of from about 500 nm to about 710 nm and at its 3′ terminus with a moiety of the following structural formula:

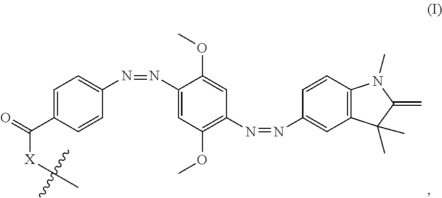

wherein:
[symbol] indicates the point of attachment of the moiety to the 3′ terminus of the probe sequence;
and
X is a linker.

Also provided herein is a kit comprising one or more (e.g., two, three) of the oligonucleotide probes described herein (e.g., an oligonucleotide probe for the N3 gene of a 20190-nCoV).

Also provided herein is a method of detecting 2019-nCoV in a sample, comprising providing a sample suspected to contain 2019-nCoV; subjecting RNA from the sample to a reverse transcription-polymerase chain reaction (RT-PCR) in the presence of an oligonucleotide probe of described herein; and detecting fluorescence from the fluorophore of the oligonucleotide probe.

The IQ-4-containing oligonucleotide probes described herein offer advantages over BHQ-1- and BHQ-2-containing probes that are currently available for the detection of 2019-nCoV. The IQ-4 oligonucleotide probes described herein are capable of detecting all three nucleocapsid gene fragments of 2019-nCoV at a very low level and enhanced detection efficiency, which has not been possible heretofore. In addition, the IQ-4-containing oligonucleotide probes described herein were found to have the lowest signal/noise ratio when compared to corresponding BHQ-1- and BHQ-2-containing probes.

### BRIEF DESCRIPTION OF THE DRAWINGS

The patent or application file contains at least one drawing executed in color. Copies of this patent or patent application publication with color drawings will be provided by the Office upon request and payment of the necessary fee.

The foregoing will be apparent from the following more particular description of example embodiments, as illustrated in the accompanying drawings.

FIG. 1 is amplification curves, and shows the average amplification for three replicates of three different quenchers for cycles 10-30 of qPCR amplification of GAPDH.

FIG. 2 shows average background noise level for three replicates of three different quenchers for cycles 1-10 of qPCR amplification of GAPDH.

FIG. 3 shows average curves for three replicates of three different quenchers for the exponential phase (cycles 14-18) of qPCR amplification of GAPDH.

FIG. 4 shows average curves for three replicates of three different quenchers for the stationary phase (cycles 20-30) of qPCR amplification of GAPDH.

FIG. 5A shows the average threshold cycle (Ct) values of qPCR using prime-probe pairs targeting the 2019 n-CoV N1 gene for three dilutions (400,000; 50,000; and 6,250 copies).

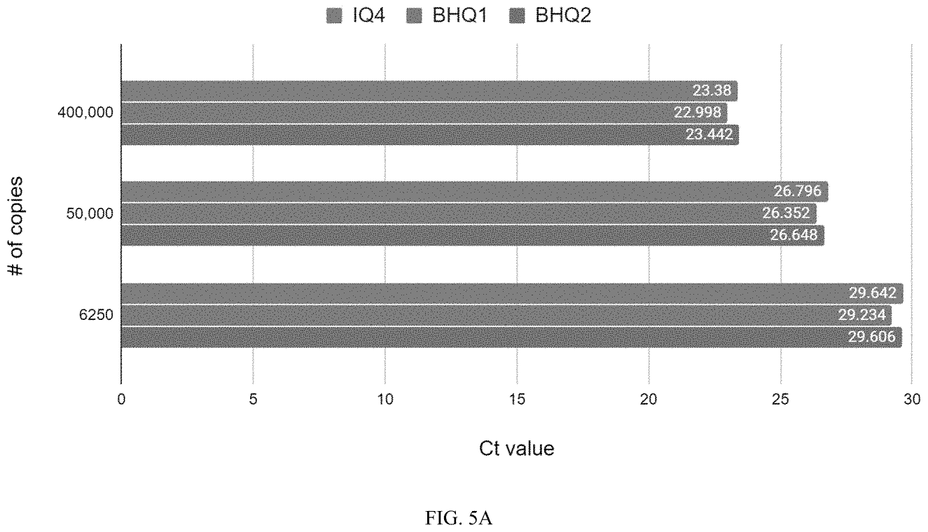

FIG. 5B shows the average Ct values of qPCR using prime-probe pairs targeting the 2019 n-CoV N2 gene for three dilutions (400,000; 50,000; and 6,250 copies).

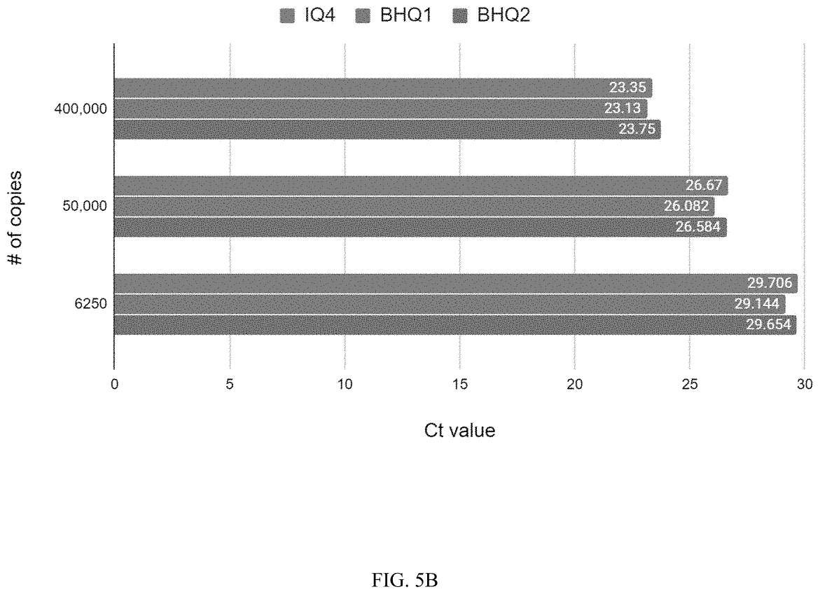

FIG. 5C shows the average Ct values of qPCR using primer-probe pairs targeting the 2019 n-CoV N3 gene for three dilutions (400,000; 50,000; and 6,250 copies).

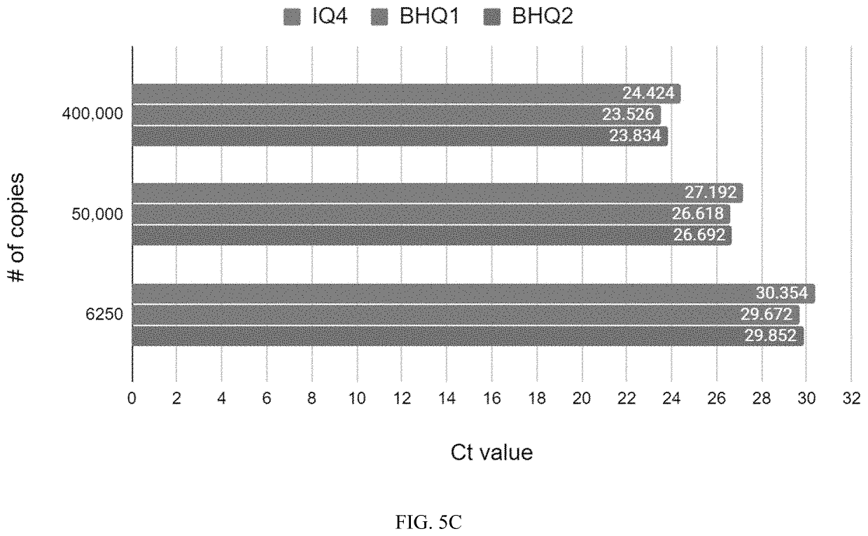

FIG. 6A shows the corrected average amplification curves of qPCR using primer probe pairs targeting the 2019-n-CoV N1 gene for three dilutions (400,000; 50,000; and 6,250 copies) and five replicates.

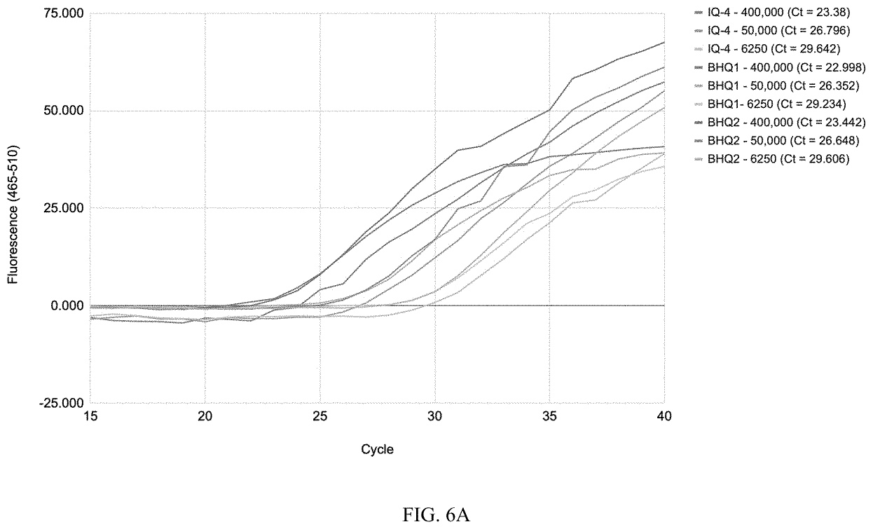

FIG. 6B shows the corrected average amplification curves of qPCR using primer probe pairs targeting the 2019-n-CoV N2 gene for three dilutions (400,000; 50,000; and 6,250 copies) and five replicates.

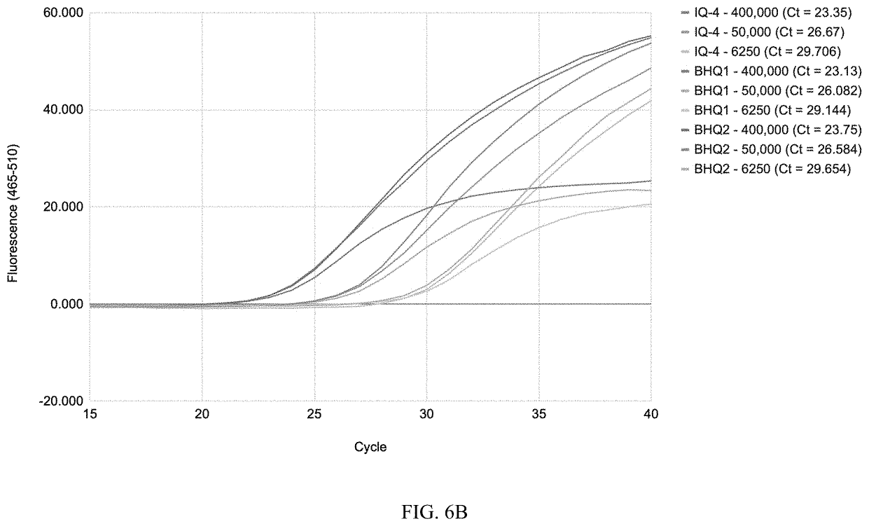

FIG. 6C shows the corrected average amplification curves of qPCR using primer probe pairs targeting the 2019-n-CoV N3 gene for three dilutions (400,000; 50,000; and 6,250 copies) and five replicates.

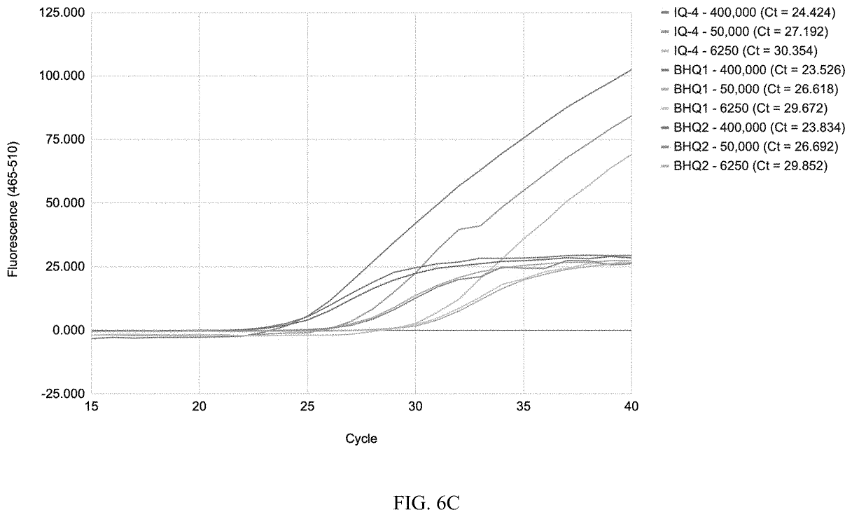

FIG. 7A shows the corrected average background noise level of qPCR using primer-probe pairs targeting the 2019-n-CoV N1 gene for three dilutions (400,000; 50,000; and 6,250 copies). The results for BHQ-1 and BHQ-2 are shown as the difference from IQ-4.

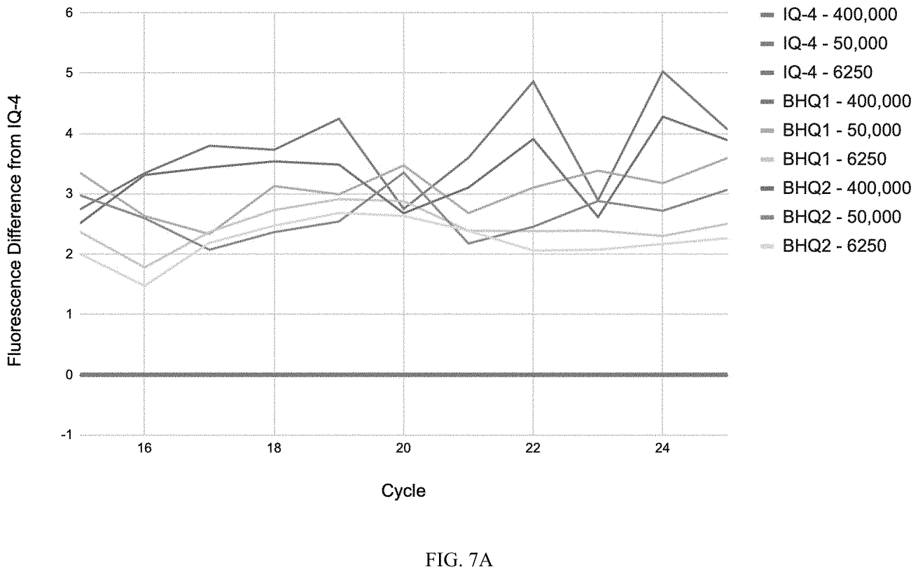

FIG. 7B shows the corrected average background noise level of qPCR using primer-probe pairs targeting the 2019-n-CoV N2 gene for three dilutions (400,000; 50,000; and 6,250 copies). The results for BHQ-1 and BHQ-2 are shown as the difference from IQ-4.

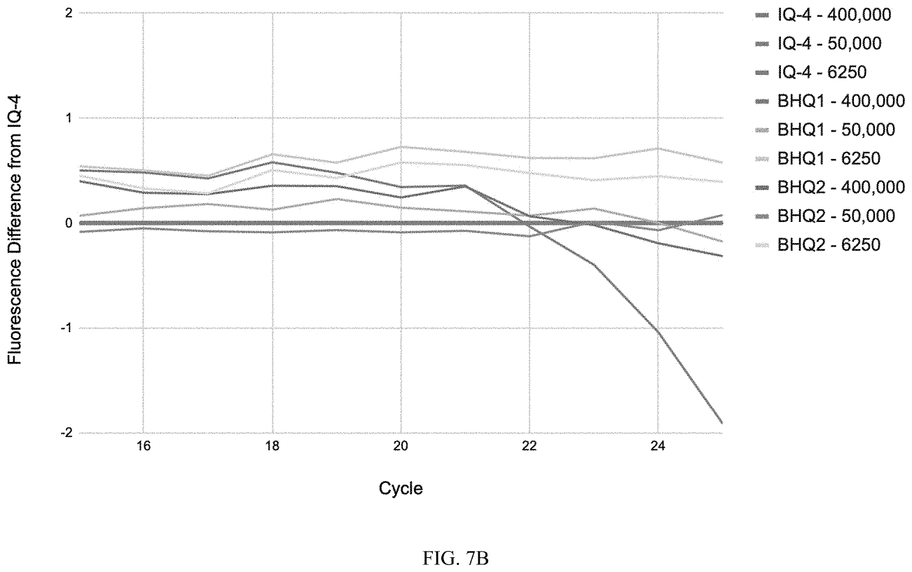

FIG. 7C shows the corrected average background noise level of qPCR using primer-probe pairs targeting the 2019-n-CoV N3 gene for three dilutions (400,000; 50,000; and 6,250 copies). The results for BHQ-1 and BHQ-2 are shown as the difference from IQ-4.

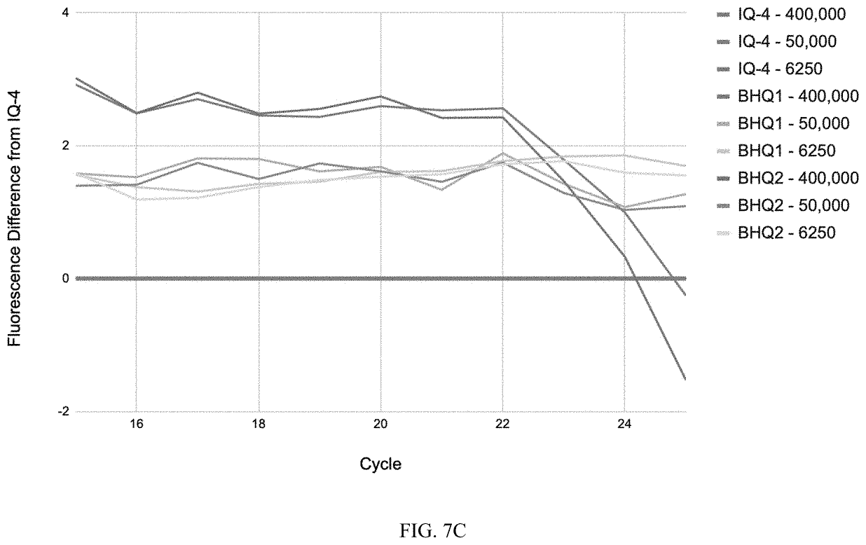

FIG. 8A shows the average Ct values of qPCR using primer-probe pairs targeting the 2019-n-CoV N1 gene for five dilutions (6,250; 781; 98; 12; and 1 copies).

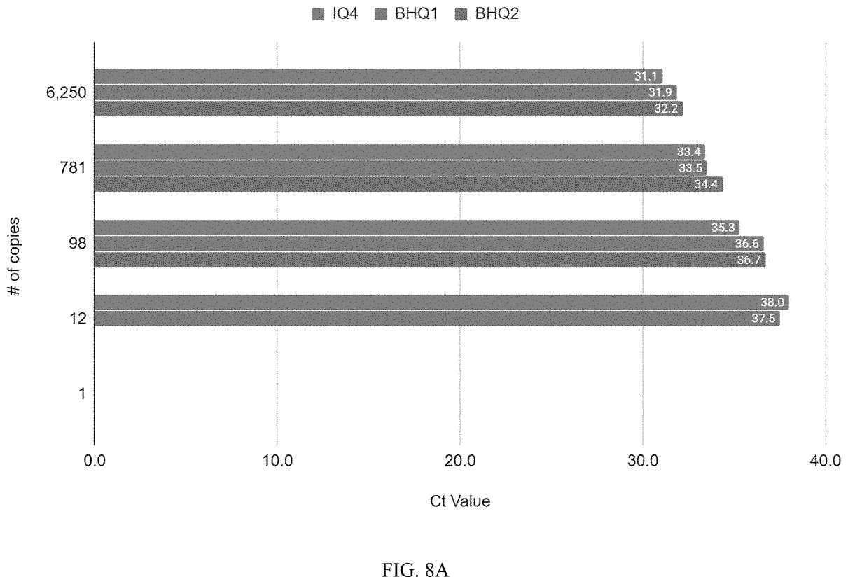

FIG. 8B shows the average Ct values of qPCR using primer-probe pairs targeting the 2019-n-CoV N2 gene for five dilutions (6,250; 781; 98; 12; and 1 copies).

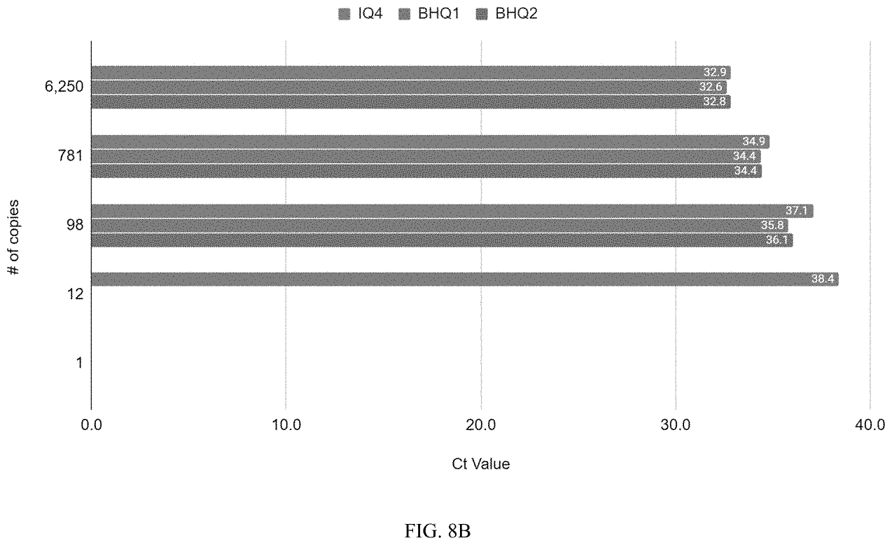

FIG. 8C shows the average Ct values of qPCR using primer-probe pairs targeting the 2019-n-CoV N3 gene for five dilutions (6,250; 781; 98; 12; and 1 copies).

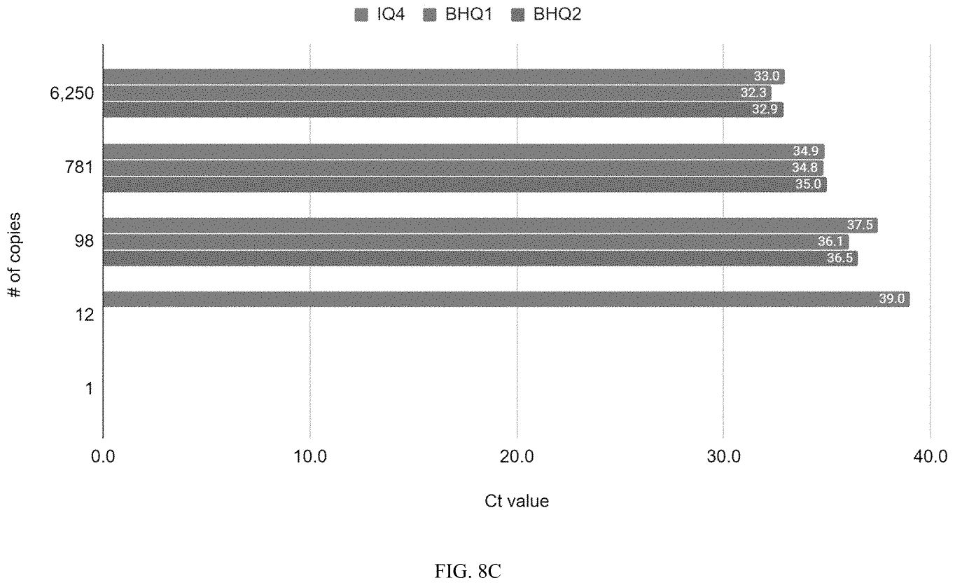

FIG. 9A shows corrected amplification curves of qPCR using primer-probe pairs targeting 2019-nCoV N1 gene for five dilutions (6,250; 781; 98; 12; and 1 copies) from triplicate reactions.

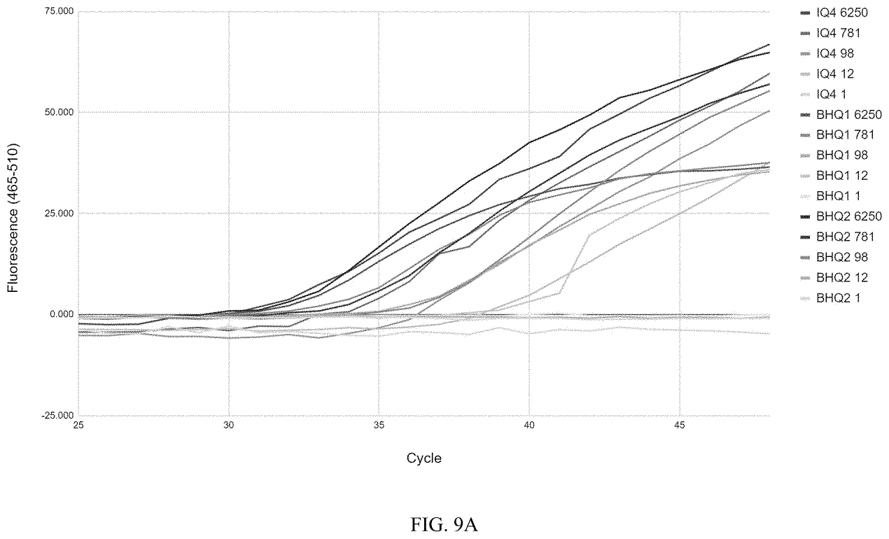

FIG. 9B shows corrected amplification curves of qPCR using primer-probe pairs targeting 2019-nCoV N2 gene for five dilutions (6,250; 781; 98; 12; and 1 copies) from triplicate reactions.

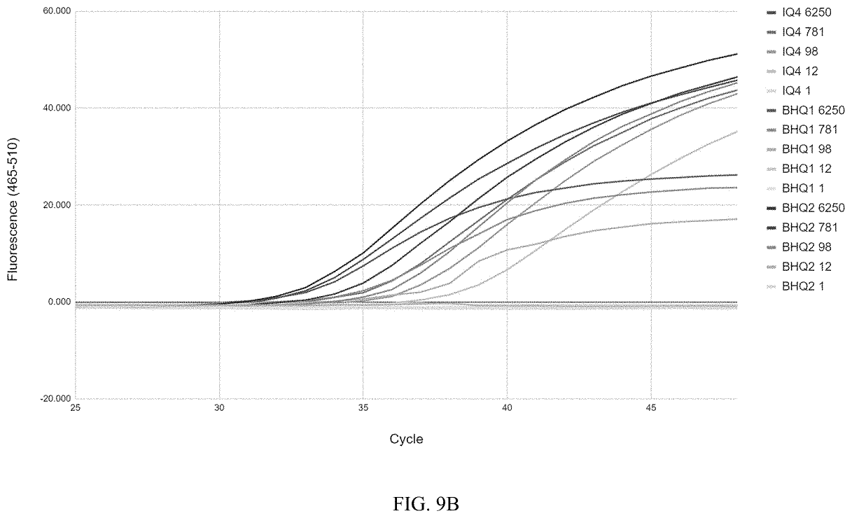

FIG. 9C shows corrected amplification curves of qPCR using primer-probe pairs targeting 2019-nCoV N3 gene for five dilutions (6,250; 781; 98; 12; and 1 copies) from triplicate reactions.

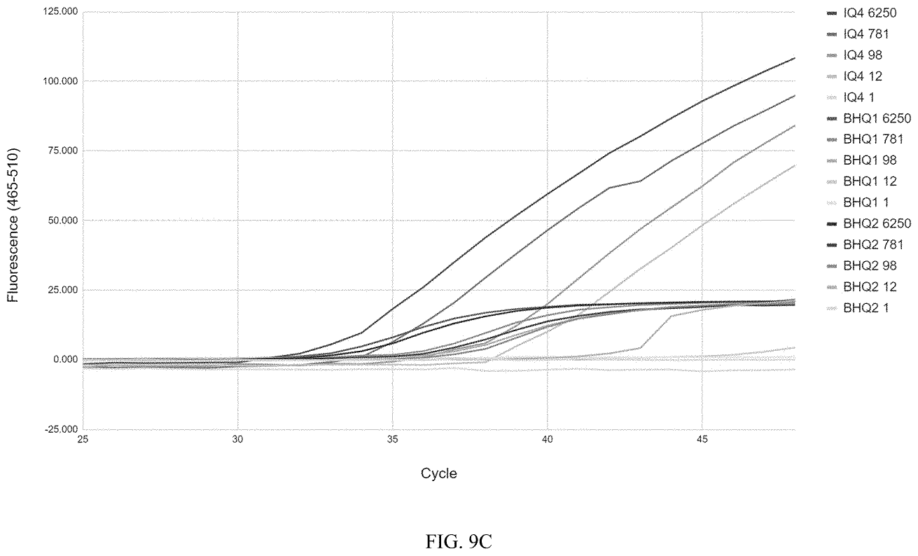

FIG. 10A is a circular map of the complete genome of the 2019-nCoV plasmid available from IDT Technologies and used in the experiments described herein.

FIG. 10B is a circular map of the nucleoplasmid gene from the 2019-nCoV plasmid available from IDT Technologies and used in the experiments described herein.

### DETAILED DESCRIPTION

A description of example embodiments follows.

### Definitions

As used herein, singular articles such as “a,” “an” and “the,” and similar referents are to be construed to cover both the singular and the plural, unless otherwise indicated herein or clearly contradicted by context. When a referent refers to the plural, the members of the plural can be the same as or different from one another. For example, reference to “a 2019-nCoV” includes a single 2019-nCoV (e.g., a single copy of 2019-nCoV, a single particle of 2019-nCoV) as well as multiple 2019-nCoV (e.g., multiple copies of 2019-nCoV, multiple particles of 2019-nCoV). In addition, the multiple 2019-nCoV may be the same as one another (e.g., genetic duplicates; of the same strain of 2019-nCoV) or different from one another (e.g., mutated variants; of multiple, different strains of 2019-nCoV), or a combination thereof (e.g., in a group of three, two are the same, but one is different).

“About” means within an acceptable error range for the particular value, as determined by one of ordinary skill in the art. Typically, an acceptable error range for a particular value depends, at least in part, on how the value is measured or determined, e.g., the limitations of the measurement system. For example, “about” can mean within an acceptable standard deviation, per the practice in the art. Alternatively, “about” can mean a range of ±20%, ±10%, ±5% or ±1% of a given value.

“Alkyl” refers to a saturated, aliphatic, branched or straight-chain, monovalent, hydrocarbon radical having the indicated number of carbon atoms, for example, from 1 to 100 carbon atoms, from 1 to 50 carbon atoms, from 1 to 25 carbon atoms, from 1 to 10 carbon atoms or, in some embodiments, from 1 to 5 carbon atoms. Thus, “(C₁-C₂₅)alkyl” means a radical having from 1-25 carbon atoms in a linear or branched arrangement. Examples of straight chain alkyl groups include, but are not limited to, methyl, ethyl, n-propyl, n-butyl, n-pentyl, n-hexyl, n-heptyl, and n-octyl. Examples of branched alkyl groups include, but are not limited to, isopropyl, iso-butyl, sec-butyl, tert-butyl, neopentyl, isopentyl, and 2,2-dimethylpropyl groups.

“Alkylene” refers to a saturated, aliphatic, branched or straight-chain, divalent, hydrocarbon radical having the indicated number of carbon atoms, for example, from 1 to 100 carbon atoms, from 1 to 50 carbon atoms, from 1 to 25 carbon atoms, from 1 to 10 carbon atoms or, in some embodiments, from 1 to 5 carbon atoms. Thus, “(C₁-C₂₅)alkylene” means a diradical having from 1-25 carbon atoms in a linear or branched arrangement. Examples of alkylene include, but are not limited to, methylene, ethylene (e.g., 1,2-ethylene, 1,1-ethylene), propylene, butylene, pentylene, and the like.

“Alkenyl” refers to an aliphatic, branched or straight-chain, monovalent, hydrocarbon radical having at least one carbon-carbon double bond and the indicated number of carbon atoms, for example, from 1 to 100 carbon atoms, from 1 to 50 carbon atoms, from 2 to 25 carbon atoms, from 2 to 10 carbon atoms or, in some embodiments, from 2 to 5 carbon atoms. Thus, “(C₂-C₂₅)alkenyl” means a radical having at least one carbon-carbon double bond and from 2 to 25 carbon atoms in a linear or branched arrangement. In some embodiments, the alkenyl group has one, two, or three carbon-carbon double bonds. Examples include, but are not limited to, vinyl, allyl, —CH═CH(CH₃), —CH═C(CH₃)₂, —C(CH₃)═CH₂, —C(CH₃)═CH(CH₃), —C(CH₂CH₃)═CH₂, among others.

“Alkenylene” refers to an aliphatic, branched or straight-chain, divalent, hydrocarbon radical having at least one carbon-carbon double bond and the indicated number of carbon atoms, for example, from 1 to 100 carbon atoms, from 1 to 50 carbon atoms, from 2 to 25 carbon atoms, from 2 to 10 carbon atoms or, in some embodiments, from 2 to 5 carbon atoms. Thus, “(C₂-C₂₅)alkenylene” means a diradical having at least one carbon-carbon double bond and from 2 to 25 carbon atoms in a linear or branched arrangement. In some embodiments, the alkenylene group has one, two, or three carbon-carbon double bonds. Alkenylene includes, but is not limited to, ethenylene and isoprenylene.

“Alkynyl” refers to an aliphatic, branched or straight-chain, monovalent, hydrocarbon radical having at least one carbon-carbon triple bond and the indicated number of carbon atoms, for example, from 1 to 100 carbon atoms, from 1 to 50 carbon atoms, from 2 to 25 carbon atoms, from 2 to 10 carbon atoms or, in some embodiments, from 2 to 5 carbon atoms. Thus, “(C₂-C₂₅)alkynyl” means a radical having at least one carbon-carbon triple bond and from 2 to 25 carbon atoms in a linear or branched arrangement. In some embodiments, the alkynyl group has one, two, or three carbon-carbon triple bonds. Examples include, but are not limited to, —C≡CH, —C≡CCH₃, —CH₂C≡CCH₃, —C≡CCH₂CH(CH₂CH₃)₂, among others.

“Alkynylene” refers to an aliphatic, branched or straight-chain, divalent, hydrocarbon radical having at least one carbon-carbon triple bond and the indicated number of carbon atoms, for example, from 1 to 100 carbon atoms, from 1 to 50 carbon atoms, from 2 to 25 carbon atoms, from 2 to 10 carbon atoms or, in some embodiments, from 2 to 5 carbon atoms. Thus, “(C₂-C₂₅)alkynylene” means a diradical having at least one carbon-carbon triple bond and from 2 to 25 carbon atoms in a linear or branched arrangement. In some embodiments, the alkynylene group has one, two, or three carbon-carbon triple bonds. Alkynylene includes, but is not limited to, propargylene.

“Heteroalkyl” refers to a saturated, branched or straight-chain, monovalent, hydrocarbon radical having the indicated number of carbon atoms, for example, from 1 to 100 carbon atoms, from 1 to 50 carbon atoms, from 1 to 25 carbon atoms, from 1 to 10 carbon atoms or, in some embodiments, from 1 to 5 carbon atoms, wherein one or more (e.g., 1, 2, 3, 4, 5 or 6) of the carbon atoms in the chain is replaced with a heteroatom selected from N, O, S and Si (e.g., N, O and S). Thus, “(C₁-C₂₅)heteroalkyl” means a radical having from 1-25 carbon atoms in a linear or branched arrangement wherein one or more (e.g., 1, 2, 3, 4, 5 or 6) of the carbon atoms in the chain is replaced with a heteroatom selected from N, O, S and Si (e.g., N, O and S). Examples of heteroalkyl groups include, but are not limited to, aminopropyl, aminocaproyl and 1-(((6-aminohexyl)carbamoyl)oxy)hexan-2-yl acetate.

“Heteroalkylene” refers to a saturated, aliphatic, branched or straight-chain, divalent, hydrocarbon radical having the indicated number of carbon atoms, for example, from 1 to 100 carbon atoms, from 1 to 50 carbon atoms, from 1 to 25 carbon atoms, from 1 to 10 carbon atoms or, in some embodiments, from 1 to 5 carbon atoms, wherein one or more (e.g., 1, 2, 3, 4, 5 or 6) of the carbon atoms in the chain is replaced with a heteroatom selected from N, O, S and Si (e.g., N, O and S). Thus, “(C₁-C₂₅)heteroalkylene” means a diradical having from 1-25 carbon atoms in a linear or branched arrangement, wherein one or more (e.g., 1, 2, 3, 4, 5 or 6) of the carbon atoms in the chain is replaced with a heteroatom selected from N, O, S and Si (e.g., N, O and S). Examples of heteroalkylene include, but are not limited to, aminopropylene, aminocaproylene and 1-(((6-aminohexyl)carbamoyl)oxy)hexan-2-ylene acetate.

“Heteroalkenyl” refers to an aliphatic, branched or straight-chain, monovalent, hydrocarbon radical having at least one carbon-carbon double bond and the indicated number of carbon atoms, for example, from 2 to 100 carbon atoms, from 2 to 50 carbon atoms, from 2 to 25 carbon atoms, from 2 to 10 carbon atoms or, in some embodiments, from 2 to 5 carbon atoms, wherein one or more (e.g., 1, 2, 3, 4, 5 or 6) of the carbon atoms in the chain is replaced with a heteroatom selected from N, O, S and Si (e.g., N, O and S). Thus, “(C₂-C₂₅)heteroalkenyl” means a radical having at least one carbon-carbon double bond and from 2 to 25 carbon atoms in a linear or branched arrangement, wherein one or more (e.g., 1, 2, 3, 4, 5 or 6) of the carbon atoms in the chain is replaced with a heteroatom selected from N, O, S and Si (e.g., N, O and S). In some embodiments, the heteroalkenyl group has one, two, or three carbon-carbon double bonds.

“Heteroalkenylene” refers to an aliphatic, branched or straight-chain, divalent, hydrocarbon radical having at least one carbon-carbon double bond and the indicated number of carbon atoms, for example, from 2 to 100 carbon atoms, from 2 to 50 carbon atoms, from 2 to 25 carbon atoms, from 2 to 10 carbon atoms or, in some embodiments, from 2 to 5 carbon atoms, wherein one or more (e.g., 1, 2, 3, 4, 5 or 6) of the carbon atoms in the chain is replaced with a heteroatom selected from N, O, S and Si (e.g., N, O and S). Thus, “(C₂-C₂₅)heteroalkenylene” means a diradical having at least one carbon-carbon double bond and from 2 to 25 carbon atoms in a linear or branched arrangement, wherein one or more (e.g., 1, 2, 3, 4, 5 or 6) of the carbon atoms in the chain is replaced with a heteroatom selected from N, O, S and Si (e.g., N, O and S). In some embodiments, the heteroalkenylene group has one, two, or three carbon-carbon double bonds.

“Heteroalkynyl” refers to an aliphatic, branched or straight-chain, monovalent, hydrocarbon radical having at least one carbon-carbon triple bond and the indicated number of carbon atoms, for example, from 2 to 100 carbon atoms, from 2 to 50 carbon atoms, from 2 to 25 carbon atoms, from 2 to 10 carbon atoms or, in some embodiments, from 2 to 5 carbon atoms, wherein one or more (e.g., 1, 2, 3, 4, 5 or 6) of the carbon atoms in the chain is replaced with a heteroatom selected from N, O, S and Si (e.g., N, O and S). Thus, “(C₂-C₂₅)heteroalkynyl” means a radical having at least one carbon-carbon triple bond and from 2 to 25 carbon atoms in a linear or branched arrangement, wherein one or more (e.g., 1, 2, 3, 4, 5 or 6) of the carbon atoms in the chain is replaced with a heteroatom selected from N, O, S and Si (e.g., N, O and S). In some embodiments, the alkynyl group has one, two, or three carbon-carbon triple bonds.

“Heteroalkynylene” refers to an aliphatic, branched or straight-chain, divalent, hydrocarbon radical having at least one carbon-carbon triple bond and the indicated number of carbon atoms, wherein one or more (e.g., 1, 2, 3, 4, 5 or 6) of the carbon atoms in the chain is replaced with a heteroatom selected from N, O, S and Si (e.g., N, O and S). Thus, “(C₂-C₂₅)heteroalkynylene” means a diradical having at least one carbon-carbon triple bond and from 2 to 25 carbon atoms in a linear or branched arrangement, wherein one or more (e.g., 1, 2, 3, 4, 5 or 6) of the carbon atoms in the chain is replaced with a heteroatom selected from N, O, S and Si (e.g., N, O and S). In some embodiments, the alkynylene group has one, two, or three carbon-carbon triple bonds.

Any of the heteroalkyl, heteroalkylene, heteroalkenyl, heteroalkenylene, heteroalkynyl and heteroalkynylene groups described herein can be unsubstituted or substituted with one or more (e.g., 1, 2, 3, 4, 5 or 6, such as 1, 2 or 3) oxo (═O), imino (═N(H) or ═N(aliphatic) or ═N(heteroaliphatic)) or thio (═S) groups. In some embodiments, a heteroalkyl, heteroalkylene, heteroalkenyl, heteroalkenylene, heteroalkynyl or heteroalkynylene is unsubstituted. In some embodiments, a heteroalkyl, heteroalkylene, heteroalkenyl, heteroalkenylene, heteroalkynyl or heteroalkynylene is substituted with one or more (e.g., 1, 2 3, 4 or 5, such as 1, 2 or 3) oxo.

Groups described herein having two or more points of attachment (i.e., divalent, trivalent, or polyvalent) within the compound of the present technology are designated by use of the suffix, “ene.” For example, divalent alkyl groups are alkylene groups, divalent heteroalkyl groups are heteroalkylene groups, and so forth. Substituted groups having a single point of attachment to the compound of the present technology are not referred to using the “ene” designation.

Additionally, unless otherwise stated, structures depicted herein are also meant to include compounds that differ only in the presence of one or more isotopically enriched atoms. For example, compounds produced by the replacement of a hydrogen with deuterium or tritium, or of a carbon with a ¹³C- or ¹⁴C-enriched carbon or of a phosphorus with a ³²P-enriched phosphorus are within the scope of this disclosure. Such compounds are useful, for example, as analytical tools, as probes in biological assays, or as therapeutic agents. Procedures for inserting such labels into the compounds of the present technology will be readily apparent to those skilled in the art based on the disclosure herein.

Those of skill in the art will appreciate that compounds of the present technology may exhibit the phenomena of tautomerism, conformational isomerism, geometric isomerism and/or stereoisomerism. Unless indicated otherwise, the present technology encompasses any tautomeric, conformational isomeric, stereochemical and/or geometric isomeric forms of the compounds having one or more of the utilities described herein, as well as mixtures of these various different forms.

Stereoisomers of compounds (also known as optical isomers) include all chiral, diastereomeric, and racemic forms of a structure, unless the specific stereochemistry is expressly indicated. Thus, compounds used in the present technology include enriched or resolved optical isomers at any or all asymmetric atoms as are apparent from the depictions. Both racemic and diastereomeric mixtures, as well as the individual optical isomers may be isolated or synthesized so as to be free or substantially free from their enantiomeric or diastereomeric partners, and these stereoisomers are all within the scope of the present technology.

### Oligonucleotide Probes

Provided herein are oligonucleotide probes for detecting a DNA or RNA molecule. The probes are particularly useful for detecting a DNA or RNA molecule by qPCR. When the probe is for detecting a DNA molecule, the probe comprises a probe sequence complementary to the sequence of the DNA molecule, or a fragment thereof. When the probe is for detecting a RNA molecule, the probe comprises a probe sequence complementary to the complementary DNA (cDNA) sequence of the sequence of the RNA molecule, or a fragment thereof. The probe sequence is modified at its 5′ terminus with a fluorophore and at its 3′ terminus with a moiety capable of quenching fluorescence from the fluorophore. The moiety has one of the following structural formulas:

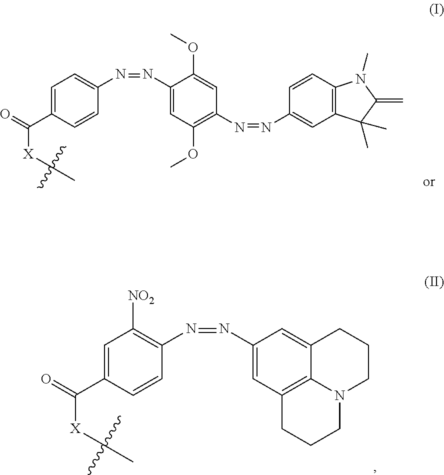

wherein:
[symbol] indicates the point of attachment of the moiety to the 3′ terminus of the probe sequence;
and
X is a linker.

For example, provided herein is an oligonucleotide probe for detecting a 2019 novel coronavirus (2019-nCoV) (e.g., the nucleocapsid, or the N1, N2 and/or N3 gene fragment thereof, of the 2019-nCoV), e.g., using RT-qPCR. The probe comprises a probe sequence complementary to the complementary DNA (cDNA) sequence of the genomic sequence of a 2019-nCoV, or a fragment thereof (e.g., the sequence of the nucleocapsid, such as the N1, N2 and/or N3 gene fragment thereof, of the 2019-nCoV, or a fragment thereof, respectively), modified at its 5′ terminus with a fluorophore and at its 3′ terminus with a moiety capable of quenching fluorescence from the fluorophore. The moiety has the structure of structural formula (I) or (II) (e.g., structural formula (I)).

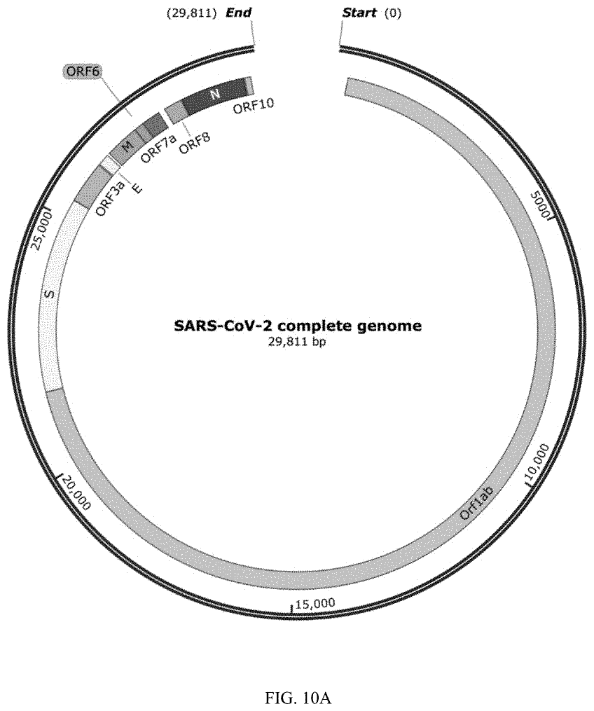

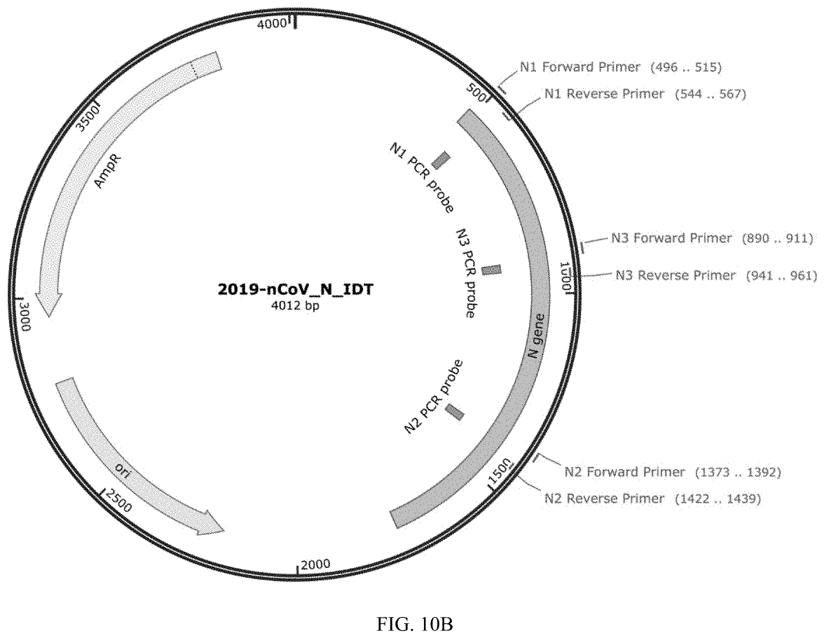

2019-nCoV is a severe acute respiratory syndrome coronavirus, likely having its origin in bats, and is the virus that causes coronavirus disease 2019 (COVID-19). 2019-nCoV has also been referred to as SARS-CoV-2. Several viral genomes of 2019-nCoV have been sequenced, and the sequences published. Many of such sequences can be found in the NCBI Virus database at https://www.ncbi.nlm.nih.gov/labs/virus/vssi/#/, including isolate Wuhan-Hu-1 (NCBI Accession No. NC 045512), which contains 29,903 nucleotides, and isolate SARS-CoV-2/human/NPL/61-TW/2020 (GenBank Accession No. MT072688.1), which contains 29,811 base pairs and forms the basis of the 2019-nCoV plasmid available from IDT Technologies and used in the experiments described herein. FIG. 10A is a circular map of the complete genome of the 2019-nCoV plasmid available from IDT Technologies and used in the experiments described herein, and shows the relative location of the nucleocapsid gene in the 2019-nCoV viral genome, various fragments of which are the target of the Diagnostic Panel.

The gene for the nucleocapsid (N) phosphoprotein of isolate Wuhan-Hu-1 (NCBI Accession No. NC 045512) extends from nucleotide number 28,274 to nucleotide number 29,533. The gene for N of isolate SARS-CoV-2/human/NPL/61-TW/2020 (GenBank Accession No. MT072688.1) extends from nucleotide number 28,259 to nucleotide number 29,518. FIG. 10B is a circular map of the nucleoplasmid gene from the 2019-nCoV plasmid available from IDT Technologies and used in the experiments described herein, and shows the relative locations of the N1, N2 and N3 genes in the N gene of 2019-nCoV. FIG. 10B also shows how the probes and primers for the N1, N2 and N3 genes of 2019-nCoV described in the Exemplification map onto the N gene and, in turn, the complete genome of 2019-nCoV.

The CDC has identified and published recommended probe and primer sequences for detection of three genes of the nucleocapsid of 2019-nCoV, N1, N2 and N3, which can be found at https://www.cdc.gov/coronavirus/2019-ncov/lab/rt-per-panel-primer-probes.html and in Tables 1 and 2. Under current CDC protocols, a sample is not considered positive for 2019-nCoV unless N1 and N2 RNA is detected in the sample.

TABLE 1
Probe Sequences For the Detection of
2019-nCoV Recommended by the CDC

| | Sequence | |
|---|---|---|
| | Description | Probe Sequence |
| SEQ ID NO: 4 | N1 Probe | ACC CCG CAT TAC GTT TGG |
| | | TGG ACC |
| SEQ ID NO: 5 | N2 Probe | ACA ATT TGC CCC CAG CGC |
| | | TTC AG |
| SEQ ID NO: 6 | N3 Probe | AYC ACA TTG GCA CCC GCA |
| | | ATC CTG |

TABLE 2
Primer Sequences For the Detection of
2019-nCoV Recommended by the CDC

| | Sequence | |
|---|---|---|
| | Description | Primer Sequence |
| SEQ ID NO: 7 | N1 Forward Primer | GAC CCC AAA ATC |
| | | AGC GAA AT |
| SEQ ID NO: 8 | N1 Reverse Primer | TCT GGT TAC TGC |
| | | CAG TTG AAT CTG |
| SEQ ID NO: 9 | N2 Forward Primer | TAA CAA ACA TTG |
| | | GCC GCA AA |
| SEQ ID NO: 10 | N2 Reverse Primer | GCG CGA CAT TCC |
| | | GAA GAA |
| SEQ ID NO: 11 | N3 Forward Primer | GGG AGC CTT GAA |
| | | TAC ACC AAA A |
| SEQ ID NO: 12 | N3 Reverse Primer | TGT AGC ACG ATT |
| | | GCA GCA TTG |

It will be appreciated that viruses, including 2019-nCoV, mutate. Thus, the sequence of 2019-nCoV and its genes may differ over time and/or from patient to patient. The oligonucleotide probes described herein can be used to detect the published sequences of 2019-nCoV and its constituent genes (e.g., the nucleoplasmid gene, or the N1, N2 and/or N3 gene fragments), or fragments thereof, as well as mutated variants of either of the foregoing. Thus, in some aspects, probe sequences described herein are those that are complementary to the cDNA sequence of the genomic sequence of a 2019-nCoV, or a fragment thereof, or a mutated variant of either of the foregoing (e.g., the sequence of SEQ ID NO: 1, 2 and/or 3, or a fragment thereof, or a mutated variant of any of the foregoing).

In some aspects, a sequence described herein (e.g., a probe sequence; the sequence of a 2019-nCoV, or a fragment thereof; a “SEQ ID NO”) has at least about 70%, e.g, at least about 75%, at least about 80%, at least about 85%, at least about 90%, at least about 95%, at least about 98% or at least about 99%, or more identity to the reference sequence.

As used herein, the term “sequence identity” means that two nucleotide or amino acid sequences, when optimally aligned, such as by the programs GAP or BESTFIT using default gap weights, share at least, e.g., 70% sequence identity, or at least 80% sequence identity, or at least 85% sequence identity, or at least 90% sequence identity, or at least 95% sequence identity, or at least 98% sequence identity, or at least about 99% sequence identity or more. For sequence comparison, typically one sequence acts as a reference sequence (e.g., parent sequence) to which test sequences are compared. The sequence identity comparison can be examined throughout the entire length of a nucleotide, or within a desired fragment of a given nucleotide. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., Current Protocols in Molecular Biology). One example of algorithm that is suitable for determining percent sequence identity and sequence similarity is the BLAST algorithm, which is described in Altschul et al., J. Mol. Biol. 215:403 (1990). Software for performing BLAST analyses is publicly available through the National Center for Biotechnology Information (publicly accessible through the National Institutes of Health NCBI internet server). Typically, default program parameters can be used to perform the sequence comparison, although customized parameters can also be used.

Sequences are “complementary” to one another when the sequences specifically hybridize to one another with consequent hydrogen bonding. Where a single polymorphism (e.g., of a 2019-nCoV, or a genomic fragment thereof) is the target for detection, then the complementarity between the oligonucleotide probe and the viral RNA should typically be exact, 100%. If less selectivity is required, then routine experimentation will determine the level of complementarity that provides the desired result (e.g., detection of a 2019-nCoV, or a genomic fragment thereof, as well as mutations of either of the foregoing, in a sample).

Typically, a fragment described herein (e.g., a fragment of a 2019-nCoV sequence, such as the sequence of the nucleocapsid, or the sequence of an N1, N2 or N3 gene) will have a length of at least 10 nucleotides, for example, at least 15 nucleotides, at least 20 nucleotides, at least 25 nucleotides, at least 50 nucleotides, from about 10 nucleotides to about 100 nucleotides, from about 10 to about 50 nucleotides, from about 10 to about 40 nucleotides or from about 10 to about 30 nucleotides.

A fragment of a specified sequence can also be described as a percentage of the length of the specified sequence. Thus, a fragment of a specified sequence described herein can contain at least 10% as many nucleotides as the specified sequence, e.g., at least 25%, at least 35%, at least 45%, at least 50%, at least 60%, at least 70%, at least 75%, at least 80%, at least 85%, at least 90% or at least 95% as many nucleotides as the specified sequence.

In some aspects, the probe sequence is complementary to the cDNA sequence of the sequence of the nucleocapsid gene of a 2019-nCoV, or a fragment thereof.

In some aspects, the probe sequence is complementary to the cDNA sequence of the sequence of the N1 gene of a 2019-nCoV, or a fragment thereof. In some aspects, the probe sequence is complementary to the cDNA sequence of the sequence of SEQ ID NO: 1, or a fragment thereof. For example, in some aspects, the probe sequence comprises, consists essentially of or consists of the sequence of SEQ ID NO: 4. In a specific aspect, the oligonucleotide probe has the sequence of SEQ ID NO: 17.

In some aspects, the probe sequence is complementary to the cDNA sequence of the sequence of the N2 gene of a 2019-nCoV, or a fragment thereof. In some aspects, the probe sequence is complementary to the cDNA sequence of the sequence of SEQ ID NO: 2, or a fragment thereof. For example, in some aspects, the probe sequence comprises, consists essentially of or consists of the sequence of SEQ ID NO: 5. In a specific aspect, the oligonucleotide probe has the sequence of SEQ ID NO: 20.

In some aspects, the probe sequence is complementary to the cDNA sequence of the sequence of the N3 gene of a 2019-nCoV, or a fragment thereof. In some aspects, the probe sequence is complementary to the cDNA sequence of the sequence of SEQ ID NO: 3, or a fragment thereof. For example, in some aspects, the probe sequence comprises, consists essentially of or consists of the sequence of SEQ ID NO: 6. In a specific aspect, the oligonucleotide probe has the sequence of SEQ ID NO: 23.

Dye pairs including a fluorophore and quencher, particularly a dark quencher (a quencher that releases energy absorbed from a fluorophore without emitting light), capable of quenching the fluorophore, have found application in a number of fields including in the detection of 2019-nCoV and diagnosis of COVID-19. A quencher is capable of quenching fluorescence from a fluorophore when light (e.g., fluorescence) emitted by the fluorophore, typically, upon irradiation of the fluorophore (e.g., at or about its absorbance maximum), can, under at least one set of conditions, be quenched by the quencher of the dye pair. The phenomenon by which a quencher quenches the fluorescence of a fluorophore is known as Forster resonance energy transfer, or FRET. Typically, quenching in FRET is accomplished when the fluorophore and quencher of the dye pair are within a certain distance (e.g., the Forster distance) of one another. In addition, there should be overlap between the emission spectrum of the fluorophore and the absorbance spectrum of the quencher. Spatial proximity for FRET can be achieved, for example, by locating individual components of a dye pair on opposing, hybridizable, self-complementary segments of a single oligonucleotide that can self-hybridize in the absence of an exogenous sequence, or by placing a quencher and fluorophore on an oligonucleotide that lacks the self-annealing property, such that the random-coil conformation of the oligonucleotide keeps the fluorophore and quencher within a suitable distance for fluorescence quenching. Upon disruption of the internal, self-hybridization, as can occur in the presence of a complementary (e.g., target) oligonucleotide, the fluorophore and quencher can be brought out of FRET range, unmasking the fluorescence of the fluorophore. A similar unmasking of a fluorophore's fluorescence can be observed upon action of the exonuclease activity of Taq polymerase in a qPCR reaction, for example, which degrades the hybridized nucleotide in the subsequent round of DNA synthesis, thereby cleaving the fluorophore and/or quencher from the probe. Selection of dye pairs can be accomplished by a person of ordinary skill in the art.

A wide variety of fluorophores suitable for use in dye pairs are known in the literature. Typically, the fluorophore is an aromatic or heteroaromatic compound, such as a pyrene, anthracene, naphthalene, acridine, stilbene, indole, benzindole, oxazole, thiazole, benzothiazole, cyanine, carbocyanine, salicylate, anthranilate, coumarin, fluorescein, rhodamine or other like compound. Examples of fluorophores suitable for use in dye pairs include xanthene dyes, such as fluorescein or rhodamine dyes, including 6-carboxyfluorescein (FAM), 2′7′-dimethoxy-4′5′-dichloro-6-carboxyfluorescein (JOE), tetrachlorofluorescein (TET), 6-carboxyrhodamine (R6G), N,N,N′,N′-tetramethyl-6-carboxyrhodamine (TAMRA) and 6-carboxy-X-rhodamine (ROX); naphthylamine dyes that have an amino group in the alpha or beta position, including 1-dimethylaminonaphthyl-5-sulfonate, 1-anilino-8-naphthalene sulfonate, 2-p-toluidinyl-6-naphthalene sulfonate and 5-(2′-aminoethyl)aminonaphthalene-1-sulfonic acid (EDANS); coumarins, such as 3-phenyl-7-isocyanatocoumarin; acridines, such as 9-isothiocyanatoacridine and acridine orange; N-(p-(2-benzoxazolyl)phenyl)maleimide; cyanines, such as indodicarbocyanine 3 (Cy3, cyanine 550), indodicarbocyanine 3.5 (Cy3.5), indodicarbocyanine 5 (Cy5, cyanine 650), indodicarbocyanine 5.5 (Cy5.5), 3-(-carboxy-pentyl)-3′-ethyl-5,5′-dimethyloxacarbocyanine (CyA); 1H,5H,11H,15H-xantheno[2,3,4-ij:5,6,7-i′j′]diquinolizin-18-ium, 9-[2(or 4)-[[[6-[2,5-dioxo-1-pyrrolidinyl)oxy]-6-oxohexyl]amino]sulfonyl]-4(or 2)-sulfophenyl]-2,3,6,7,12,13,16,17-octahydro-inner salt (TR or Texas Red); BODIPY dyes, such as BODIPY R6G and BODIPY TMR; benzooxazoles; stilbenes; pyrenes; Alexa Fluor® dyes (available from ThermoFisher Scientific); DyLight® dyes (available from ThermoFisher Scientific); Quasar® dyes; Pulsar® dyes; quantum dots, and the like. In some aspects, the fluorophore is FAM, Oregon Green (4-(2,7-difluoro-6-hydroxy-3-oxo-3H-xanthen-9-yl)isophthalic acid), Rhodamine Green (carboxyrhodamine 110), TET, Cal Fluor® Gold (1-[2-(6-ethylamino-2, 7-dimethyl-3-oxo-3H-xanthen-9-yl)-benzoic acid), BODIPY R6G (3-(4,4-difluoro-5-phenyl-3a,4a-diaza-4-bora-s-indacen-3-yl)propionic acid), Yakima Yellow, JOE, HEX, Cal Orange, BODIPY TMR (3-[4,4-difluoro-5-(p-methoxyphenyl)-1,3-dimethyl-3a,4a-diaza-4-bora-s-indacen-2-yl]propionic acid), Quasar-570 (indo-3-carbocyanine N-ethyl-N′-hexanoic acid), Cy3 (1-{6-[(2,5-Dioxo-1-pyrrolidinyl)oxy]-6-oxohexyl}-2-[(1E,3E)-3-(1-{6-[(2,5-dioxo-1-pyrrolidinyl)oxy]-6-oxohexyl}-3,3-dimethyl-5-sulfo-1,3-dihydro-2H-indol-2-ylidene)-1-propen-1-yl]-3,3-dimethyl-3H-indolium-5-sulfonate), TAMRA, Rhodamine Red-X (54(5-carboxypentyl)sulfamoyl]-2-[6-(diethylamino)-3-(diethyliminio)-3H-xanthen-9-yl]benzenesulfonate), Redmond Red, Cy3.5, CROX, Cal Red, Texas Red, Pulsar or Cy5.5. In some aspects, the fluorophore is fluorescein, tetrachlorofluorescein, hexachlorofluorescein, Cy3, TAMRA, Cy3.5, carboxy-x-rhodamine, Texas Red or Cy5. In a specific aspect, the fluorophore is fluorescein.

Emission maxima of selected fluorophores are listed in the following table:

| Fluorophore | Emission Max |
|---|---|
| Fluorescein | 520 nm |
| Tetrachloro fluorescein (TET) | 536 nm |
| Hexachlorofluorescein (HEX) | 556 nm |
| Cy3 | 570 nm |
| Tetramethylrhodamine (Tamra) | 580 nm |
| Cy3.5 | 596 nm |
| Carboxy-x-rhodamine (Rox) | 605 nm |
| Texas Red | 610 nm |
| Cy5 | 667 nm |
| Cy5.5 | 694 nm |

In some aspects, the moiety that quenches the fluorescence of the fluorophore is an IQ-4-based quencher, e.g., having the structure of structural formula (I). IQ-4 corresponds to a quencher of the following structure:

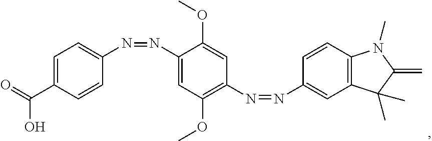 [SMILES: C=C1N(C)c2ccc(/N=N\c3cc(OC)c(/N=N\c4ccc(C(=O)O)cc4)cc3OC)cc2C1(C)C]

and is available from ChemGenes Corporation. The IQ-4 quencher is capable of absorbing fluorescent energy in the range of about 500 nm to about 725 nm (e.g., about 520 nm to about 706 nm). Accordingly, in some aspects, the fluorophore for use in an oligonucleotide probe containing an IQ-4-based quencher has an emission maximum of from about 500 nm to about 725 nm, e.g., from about 520 nm to about 706 nm or from about 500 nm to about 660 nm. Representative fluorophores meeting these criteria include fluorescein, tetrachlorofluorescein, hexachlorofluorescein, Cy3, tetramethylrhodamine, Cy 3.5, carboxy-x-rhodamine, Texas Red and Cy5. Methods of synthesizing IQ-4 and derivatizing IQ-4 for incorporation into an oligonucleotide, e.g., at the 5′ terminus of an oligonucleotide synthesized on a solid support, are described in U.S. Pat. No. 7,956,169, the entire content of which is incorporated herein by reference. Methods of elaborating an IQ-4-derivatized solid support, e.g., with an oligonucleotide, such as a FAM-modified oligonucleotide, are within the abilities of a person skilled in the art in view of the present disclosure.

In some aspects, the quencher is an IQ-2-based quencher, e.g., having the structure of structural formula (II). IQ-2 corresponds to a chromophore of the following structure:

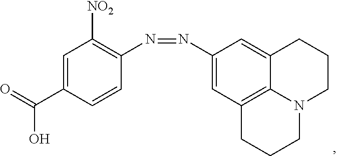 [SMILES: O=C(O)c1ccc(/N=N\c2cc3c4c(c2)CCCN4CCC3)c([N+](=O)[O-])c1]

and is available from ChemGenes Corporation. The IQ-2 quencher is capable of absorbing fluorescent energy in the range of about 420 nm to about 600 nm. Accordingly, in some aspects, the fluorophore for use in an oligonucleotide probe containing an IQ-2-based quencher has an emission maximum of from about 420 nm to about 600 nm. Methods of synthesizing IQ-2 and derivatizing IQ-2 for incorporation into an oligonucleotide, e.g., at the 5′ terminus of an oligonucleotide synthesized on a solid support, are described in U.S. Pat. No. 8,530,634, the entire content of which is incorporated herein by reference. Methods of elaborating an IQ-2-derivatized solid support, e.g., with an oligonucleotide, such as a FAM-modified oligonucleotide, are within the abilities of a person skilled in the art in view of the present disclosure.

Fluorescence is quenched herein when the dye pair is in a configuration in which the intensity of the fluorescence signal from the fluorophore in the absence of quenching is reduced by the presence of the quencher by at least 50%, e.g., at least 60%, at least 70%, at least 75%, at least 80%, at least 85%, at least 90%, at least 95%, at least 98% or at least 99%. High levels of quenching allow for the preparation of oligonucleotide probes having a high signal to noise ratio, which is the ratio of the intensity of the fluorescence signal present (e.g., at a particular wavelength or range of wavelengths) when the composition is in its maximally unquenched state (signal) to the intensity of the fluorescence signal present (e.g., at the particular wavelength or range of wavelengths) when the composition is in its maximally quenched state (noise). Probes having a high signal to noise ratio are desirable for the development of highly sensitive assays.

To measure signal to noise ratios, fluorescence is measured (e.g., at a particular wavelength or range of wavelengths) in a configuration in which the quencher and fluorophore are within the Forster distance and the fluorophore is maximally quenched, and compared to fluorescence measured (e.g., at the particular wavelength or range of wavelengths) when fluorophore and quencher are separated in the absence of quenching. The signal to noise ratio of a dye pair is generally at least about 2:1, but is preferably higher, e.g., at least about 3:1, at least about 4:1, at least about 5:1 or at least about 10:1. Signal to noise ratios can be affected by the fluorophore-quencher pair, the quality of the synthesis, and the oligonucleotide sequence.

Linkers suitable for use in the oligonucleotide probes described herein include those chemical groups that can connect a quencher to the 3′ terminus of the probe sequence and are stable under the conditions associated with the intended use of the oligonucleotide probe, e.g., qPCR. To provide the requisite connection, a linker will typically be at least difunctional, e.g., will contain at least two reactive groups that facilitate attachment of the linker to the quencher, on the one hand, and the probe sequence, on the other. More typically, however, the linker will be trifunctional, so as to facilitate solid-phase synthesis of the oligonucleotide probe. A linker designed to facilitate solid-phase synthesis of the oligonucleotide probe will also be cleavable from the solid phase upon completion of the synthesis, to facilitate release of the oligonucleotide probe from the solid phase. Examples of suitable linkers include those described in U.S. Pat. Nos. 7,956,169 and 8,530,634, the entire contents of which are incorporated herein by reference. Further examples of linkers include (C₁-C₁₀₀)alkylene, (C₁-C₁₀₀)alkenylene, (C₁-C₁₀₀)alkynylene, (C₁-C₁₀₀)heteroalkylene, (C₁-C₁₀₀)heteroalkenylene and (C₁-C₁₀₀)heteroalkynylene (e.g., (C₁-C₂₅)alkylene, (C₁-C₂₅)alkenylene, (C₁-C₂₅)alkynylene, (C₁-C₂₅)heteroalkylene, (C₁-C₂₅)heteroalkenylene and (C₁-C₂₅)heteroalkynylene), and

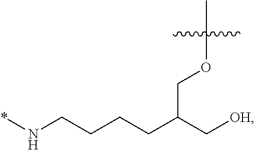 [SMILES: *NCCCCC(CO)CO*]

wherein * indicates the point of attachment of X to the carbonyl of structural formula I or II (e.g., structural formula (I)).

Thus, in some aspects, X is (C₁-C₂₅)alkylene, (C₁-C₂₅)alkenylene, (C₁-C₂₅)alkynylene, (C₁-C₂₅)heteroalkylene, (C₁-C₂₅)heteroalkenylene or (C₁-C₂₅)heteroalkynylene (e.g. (C₁-C₂₅)alkylene or (C₁-C₂₅)heteroalkylene). In some more specific aspects, X is

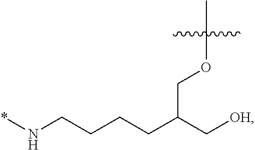 [SMILES: *NCCCCC(CO)CO*]

wherein * indicates the point of attachment of X to the carbonyl of structural formula I or II (e.g., structural formula (I)).

Methods of synthesizing oligonucleotide probes, such as the oligonucleotide probes described herein, are within the abilities of a person skilled in the art.

### Kits

Also provided herein are kits for detecting a 2019-nCoV and/or diagnosing COVID-19 using one or more (e.g., two or three) oligonucleotide probes described herein. Thus, provided herein is a kit comprising one or more (e.g., two or three) oligonucleotide probes described herein (e.g., an oligonucleotide probe wherein the probe sequence is complementary to the cDNA sequence of the sequence of a 2019-nCoV, or a fragment thereof, such as the sequence of the N1, N2 or N3 gene, or a fragment thereof). One aspect is a kit comprising: (i) an oligonucleotide probe wherein the probe sequence is complementary to the cDNA sequence of the sequence of the N3 gene of a 2019-nCoV, or a fragment thereof and (ii) an oligonucleotide probe wherein the probe sequence is complementary to the cDNA sequence of the sequence of the N1 or N2 gene of a 2019-nCoV, or a fragment thereof. Another aspect is a kit comprising: (i) an oligonucleotide probe wherein the probe sequence is complementary to the cDNA sequence of the sequence of the N3 gene of a 2019-nCoV, or a fragment thereof (e.g., the sequence of SEQ ID NO: 3, or a fragment thereof); (ii) an oligonucleotide probe wherein the probe sequence is complementary to the cDNA sequence of the N2 gene of the 2019-nCoV, or a fragment thereof (e.g., the sequence of SEQ ID NO: 2, or a fragment thereof); and (iii) an oligonucleotide probe wherein the probe sequence is complementary to the cDNA sequence of the N1 gene of the 2019-nCoV, or a fragment thereof (e.g., the sequence of SEQ ID NO: 1, or a fragment thereof).

The oligonucleotide probes and kits described herein are useful as probes and kits for qPCR. Thus, in some aspects, the kit further comprises a forward primer and a reverse primer for the target of one or more (e.g., each) of the oligonucleotide probes in the kit (e.g., a cDNA sequence of the sequence of a 2019-nCoV; the N gene of a 2019-nCoV; the N1, N2 and/or N3 gene of a 2019-nCoV, or a fragment thereof). In some aspects, the kit further comprises a forward primer and a reverse primer for the cDNA sequence of the sequence of the N1 gene, or a fragment thereof. In some aspects, the kit further comprises a forward primer and a reverse primer for the cDNA sequence of the sequence of the N2 gene, or a fragment thereof. In some aspects, the kit further comprises a forward primer and a reverse primer for the cDNA sequence of the sequence of the N3 gene, or a fragment thereof.

In an aspect of a kit comprising an oligonucleotide probe for the N1 gene of a 2019-nCoV, the forward primer comprises, consists essentially of or consists of the sequence of SEQ ID NO: 7, and the reverse primer comprises, consists essentially of or consists of the sequence of SEQ ID NO: 8.

In an aspect of a kit comprising an oligonucleotide probe for the N2 gene of a 2019-nCoV, the forward primer comprises, consists essentially of or consists of the sequence of SEQ ID NO: 9, and the reverse primer comprises, consists essentially of or consists of the sequence of SEQ ID NO: 10.

In an aspect of a kit comprising an oligonucleotide probe for the N3 gene of a 2019-nCoV, the forward primer comprises, consists essentially of or consists of the sequence of SEQ ID NO: 11, and the reverse primer comprises, consists essentially of or consists of the sequence of SEQ ID NO: 12.

In some aspects, a kit further comprises an enzyme (e.g., DNA polymerase and/or reverse transcriptase). LunaScript™ RT SuperMix (New England BioLabs, Catalog No. E3010) is a convenient source of reverse transcriptase. Luna® Universal Probe PCR MasterMix (New England BioLabs, Catalog No. M3004) is a convenient source of DNA polymerase.

In some aspects, a kit further comprises one or more control probes and, optionally, a forward primer and a reverse primer for the target of a control probe. Examples of targets of a control probe include GAPDH and BRCA (e.g., BRCA1).

In some aspects, a kit further comprises diethylpyrocarbonate-treated (DEPC) water.

In a specific aspect, a kit comprises a first control probe for a GAPDH gene (e.g., having the sequence of SEQ ID NO:14), a forward primer for the GAPDH gene (e.g., having the sequence of SEQ ID NO:3), a reverse primer for the GAPDH gene (e.g., having the sequence of SEQ ID NO:13), a second control probe for a BRCA1 gene (e.g., having the sequence of SEQ ID NO:1), a forward primer for the BRCA1 gene (e.g., having the sequence of SEQ ID NO:26), a reverse primer for the BRCA1 gene (e.g., having the sequence of SEQ ID NO:27), Total RNA (human) (e.g., available from ThermoFisher, Catalog No. 4307281), LunaScript™ RT SuperMix, Luna® Universal Probe PCR MasterMix, DEPC water, no RT control mix, an oligonucleotide probe for the N1 gene of a 2019-nCoV (e.g., having the sequence of SEQ ID NO:17), a forward primer for the cDNA sequence of the sequence of the N1 gene of the 2019-nCoV (e.g., having the sequence of SEQ ID NO:7), a reverse primer for the cDNA sequence of the sequence of the N1 gene of the 2019-nCoV (e.g., having the sequence of SEQ ID NO:8), an oligonucleotide probe for the N2 gene of a 2019-nCoV (e.g., having the sequence of SEQ ID NO:20), a forward primer for the cDNA sequence of the sequence of the N2 gene of the 2019-nCoV (e.g., having the sequence of SEQ ID NO:9), a reverse primer for the cDNA sequence of the sequence of the N2 gene of the 2019-nCoV (e.g., having the sequence of SEQ ID NO:10), an oligonucleotide probe for the N3 gene of a 2019-nCoV (e.g., having the sequence of SEQ ID NO:23), a forward primer for the cDNA sequence of the sequence of the N3 gene of the 2019-nCoV (e.g., having the sequence of SEQ ID NO:11) and a reverse primer for the cDNA sequence of the sequence of the N3 gene of the 2019-nCoV (e.g., having the sequence of SEQ ID NO:12),

The kits provided herein can be adapted to singleplex and multiplex qPCR applications. In singleplex qPCR, a single gene, either a gene of interest or a control, is amplified in each qPCR well. If a qPCR for a gene of interest and a qPCR for a control are each performed in triplicate, the sample will have to be divided into six wells—three for the gene of interest and three for the control gene. In multiplex qPCR, two or more target genes are amplified in the same reaction, using the same reagent mix. Using multiplexing, the amount of sample required for a qPCR reaction can be reduced because the expression of more than one gene can be measured in a single reaction. Multiplex analysis is as sensitive and accurate as single-gene amplification or singleplex, but can be more technically complex. Probes based on

It will be appreciated that in order to detect and/or quantify each gene in a multiplex analysis individually, each gene must be matched to an independently-detectable fluorophore. Typically, this is achieved by selecting fluorophores having sufficiently different wavelengths of absorption and/or emission that they can be differentiated from one another. Ideally, there is little overlap between the absorption and/or emission spectra of independently-detectable fluorophores, so that analysis of one does not skew analysis of the other to a detectable degree. The use of independently-detectable fluorophores may require use of different quenchers (e.g., to form a suitable dye pair). Thus, in some aspects of a kit comprising two or more oligonucleotide probes, the probes contain independently-detectable fluorophores. Examples of independent-detectable fluorophores include Cy3 and Cy5, Cy3.5 and Cy5.5, FAM and Cy5 or Cy5.5.

### Detection/Diagnostic Methods

Also provided herein are methods of detecting a 2019-nCoV in a sample and/or methods of diagnosing COVID-19 in a subject (e.g., from a sample from the subject) using one or more (e.g., two, three) oligonucleotide probes described herein (e.g., an oligonucleotide probe for the N1 gene of a 2019-nCoV, an oligonucleotide probe for the N2 gene of the 2019-nCoV and/or an oligonucleotide probe for the N3 gene of the 2019-nCoV). The methods of detecting a 2019-nCoV in a sample can be used to diagnose COVID-19 in a subject by detecting 2019-nCoV in a sample from the subject.

One aspect comprises providing a sample suspected to contain a 2019-nCoV; subjecting RNA from the sample to a reverse transcription-polymerase chain reaction (RT-PCR) in the presence of an oligonucleotide probe described herein; and detecting fluorescence from the fluorophore of the oligonucleotide probe. In some further aspects, the presence of fluorescence from the fluorophore of the oligonucleotide probe indicates the sample contains the 2019-nCoV.

It will be appreciated that the probes, kits and methods described herein can be general to multiple, all or substantially all (e.g., the majority of, at least 50%, at least 60%, at least 75%, at least 80%, at least 85%, at least 90%, at least 95%, at least 98% or at least 99% of) variants (e.g., mutants, strains) of 2019-nCoV, or can be made specific to a single variant of the virus. Methods adapted to detection of variants actually circulating or suspected in the sample and/or subject or population of samples and/or subjects to be tested will be particularly useful. Thus, the methods described herein can be used to detect one or more different 2019-nCoV. When there is more than one different 2019-nCoV in a sample and/or subject, presence of fluorescence indicates the presence of at least one of the more than one different 2019-nCoV in the sample and/or subject. Adaptation of the methods described herein to the desired set or subset of 2019-nCoV variant(s) to be detected can be accomplished by manipulating the probe and/or primer sequences used to perform the methods described herein (e.g., the specificity and/or selectivity of the probe and/or primer sequences).

Another aspect comprises providing a sample suspected to contain a 2019-nCoV. RNA from the sample is subjected to a first RT-PCR in the presence of a first oligonucleotide probe (e.g., an oligonucleotide probe for the N3 gene of a 2019-nCoV), and fluorescence from the fluorophore of the first oligonucleotide probe is detected. RNA from the sample is subjected to a second RT-PCR in the presence of a second oligonucleotide probe (e.g., an oligonucleotide probe for the N1 or N2 gene of the 2019-nCoV), and fluorescence from the fluorophore of the second oligonucleotide probe is detected. In some aspects, the second oligonucleotide probe is an oligonucleotide probe for the N2 gene of a 2019-nCoV, and the method further comprises subjecting RNA from the sample to a third RT-PCR in the presence of a third oligonucleotide probe (e.g., an oligonucleotide probe for the N1 gene of the 2019-nCoV), and detecting fluorescence from the fluorophore of the third oligonucleotide probe. These aspects are particularly useful for conducting singleplex qPCR analyses of a sample.

Another aspect comprises providing a sample suspected to contain 2019-nCoV. RNA from the sample is subjected to RT-PCR in the presence of a first oligonucleotide probe (e.g., an oligonucleotide probe for the N3 gene of the 2019-nCoV) and a second oligonucleotide probe (e.g., an oligonucleotide probe for the N1 or N2 gene of the 2019-nCoV), wherein the fluorophores of the first oligonucleotide probe and the second oligonucleotide probe are independently-detectable. Fluorescence from the fluorophore of the first oligonucleotide probe and the fluorophore of the second oligonucleotide probe from the RT-PCR is detected. In some aspects, RNA from the sample is subjected to RT-PCR in the presence of a first oligonucleotide probe (e.g., an oligonucleotide probe for the N3 gene of the 2019-nCoV), a second oligonucleotide probe (e.g., an oligonucleotide probe for the N2 gene of the 2019-nCoV) and a third oligonucleotide probe (e.g., an oligonucleotide probe for the N1 gene of the 2019-nCoV), wherein the fluorophore of the first oligonucleotide probe, the fluorophore of the second oligonucleotide probe and the fluorophore of the third oligonucleotide probe are independently-detectable. Fluorescence from the fluorophore of the first oligonucleotide probe, the fluorophore of the second oligonucleotide probe and the fluorophore of the third oligonucleotide probe is detected. These aspects are particularly useful for conducting multiplex qPCR analyses of a sample.

In methods involving first and second oligonucleotide probes, the presence of fluorescence from the fluorophore of either oligonucleotide probe in the RT-PCR (e.g., the first oligonucleotide probe and/or the second oligonucleotide probe) indicates, in some aspects, the sample contains the 2019-nCoV. In other aspects of methods involving first and second oligonucleotide probes, the presence of fluorescence from the fluorophore of both of the oligonucleotide probes (e.g., the first oligonucleotide probe and the second oligonucleotide probe) indicates the sample contains the 2019-nCoV; the absence of fluorescence from the fluorophore of both oligonucleotide probes (e.g., the first oligonucleotide probe and the second oligonucleotide probe) indicates the sample does not contain the 2019-nCoV; and the presence of fluorescence from the fluorophore of one oligonucleotide probe (e.g., the first oligonucleotide probe, but not the second oligonucleotide probe, or the second oligonucleotide probe, but not the first oligonucleotide probe) indicates the method is inconclusive with respect to the presence or absence of the 2019-nCoV in the sample.

In methods involving first, second and third oligonucleotide probes, the presence of fluorescence from the fluorophore of any one oligonucleotide probe (e.g., the first oligonucleotide probe, the second oligonucleotide probe and/or the third oligonucleotide probe) indicates, in some aspects, the sample contains the 2019-nCoV. In other aspects of methods involving first, second and third oligonucleotide probes, the presence of fluorescence from the fluorophore of two or three of the oligonucleotide probes (e.g., the first, second and third oligonucleotide probes) indicates the sample contains the 2019-nCoV; and the absence of fluorescence from the fluorophore of two or three of the oligonucleotide probes indicates the sample does not contain the 2019-nCoV.

It will be understood that “presence of fluorescence,” used herein, refers to a fluorescence signal that is above the noise, limit of detection and/or background associated with the method and/or instrument of detection. Conversely, “absence of fluorescence,” used herein, refers to a fluorescence signal that falls below or within the noise, limit of detection and/or background level associated with the method and/or instrument of detection.

In some aspects, the sample is from a human. In some aspects, the sample is a nasal, nasopharyngeal, oropharyngeal, sputum, lower respiratory tract, bronchoalveolar or stool sample (e.g., a nasopharyngeal, oropharyngeal or sputum sample), such as a nasal, nasopharyngeal, oropharyngeal, sputum, lower respiratory tract, bronchoalveolar or stool sample from a human. Nasal, nasopharyngeal and oropharyngeal samples can conveniently be obtained by taking a swab of the indicated area or sample. Nasal, nasopharyngeal and lower respiratory tract samples can be obtained from aspirates and/or washes. A sputum sample can be obtained by spitting, for example. A broncoalveolar sample can be obtained by lavage. In some aspects, the sample is a nasopharyngeal sample (e.g., a nasopharyngeal sample from a human). In some aspects, the sample is an oropharyngeal sample (e.g., an oropharyngeal sample from a human). In some aspects the sample is a sputum sample (e.g., a sputum sample from a human).

There are several commercially available, real-time and, in some cases, validated, PCR instruments. The large capacity (>96-microwell format) instruments, which include the ABI Prism series (7000, 7300, and 7500), the MyiQ and iCycler, Mx4000, MX3000p, Chromo4, Opticon, Opticon 2 and SynChron, may be particularly useful in laboratories with large numbers of specimens. However, thermocycling on these instruments is typically slower than on lower-capacity instruments, which include the LightCycler 1.0, LightCycler 2.0 and SmartCycler II. The slower thermocycling is due to the use of a solid-phase material for heat conductance (heating block principle). The large-capacity instruments support high-volume testing while the rapid, lower-capacity instruments permit work flow flexibility that may be especially useful for laboratories that test fewer samples. Selection of an appropriate PCR instrument for the workload and work flow of a particular laboratory is within the abilities of a person of ordinary skill in the art.

### Embodiments

1. An oligonucleotide probe for detecting a 2019 novel coronavirus (2019-nCoV), comprising a probe sequence complementary to the complementary DNA (cDNA) sequence of the sequence of a N1, N2 or N3 gene of the 2019-nCoV, or a fragment thereof, and modified at its 5′ terminus with a fluorophore having an emission maximum of from about 500 nm to about 710 nm and at its 3′ terminus with a moiety of the following structural formula:

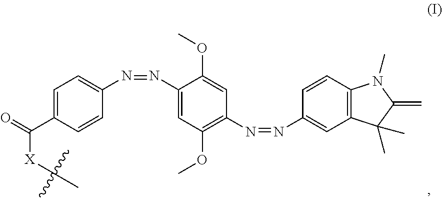

wherein:
[symbol] indicates the point of attachment of the moiety to the 3′ terminus of the probe sequence;
and
X is a linker.
2. The oligonucleotide probe of embodiment 1, wherein the probe sequence is complementary to the cDNA sequence of the sequence of the N1 gene of the 2019-nCoV, or a fragment thereof.
3. The oligonucleotide probe of embodiment 1, wherein the probe sequence is complementary to the cDNA sequence of the sequence of the N2 gene of the 2019-nCoV, or a fragment thereof.
4. The oligonucleotide probe of embodiment 1, wherein the probe sequence is complementary to the cDNA sequence of the sequence of the N3 gene of the 2019-nCoV, or a fragment thereof.
5. The oligonucleotide probe of embodiment 1 or 2, wherein the probe sequence comprises the sequence of SEQ ID NO: 4.
6. The oligonucleotide probe of embodiment 5, wherein the probe sequence consists of the sequence of SEQ ID NO: 4.
7. The oligonucleotide probe of embodiment 1 or 3, wherein the probe sequence comprises the sequence of SEQ ID NO: 5.
8. The oligonucleotide probe of embodiment 7, wherein the probe sequence consists of the sequence of SEQ ID NO: 5.
9. The oligonucleotide probe of embodiment 1 or 4, wherein the probe sequence comprises the sequence of SEQ ID NO: 6.
10. The oligonucleotide probe of embodiment 9, wherein the probe sequence consists of the sequence of SEQ ID NO: 6.
11. The oligonucleotide probe of any one of embodiments 1-10, wherein the fluorophore is fluorescein, tetrachlorofluorescein, hexachlorofluorescein, Cy3, tetramethylrhodamine, Cy 3.5, carboxy-x-rhodamine, Texas Red or Cy5.
12. The oligonucleotide probe of embodiment 11, wherein the fluorophore is fluorescein.
13. The oligonucleotide probe of any one of embodiments 1-12, wherein X is (C₁-C₂₅)alkylene, (C₁-C₂₅)alkenylene, (C₁-C₂₅)alkynylene, (C₁-C₂₅)heteroalkylene, (C₁-C₂₅)heteroalkenylene or (C₁-C₂₅)heteroalkynylene (e.g., (C₁-C₂₅)heteroalkylene).
14. The oligonucleotide probe of embodiment 13, wherein X is

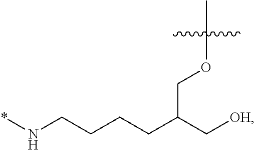 [SMILES: *NCCCCC(CO)CO*]

wherein * indicates the point of attachment of X of the carbonyl of structural formula I.
15. The oligonucleotide probe of embodiment 1, having the sequence of SEQ ID NO: 17.
16. The oligonucleotide probe of embodiment 1, having the sequence of SEQ ID NO: 20.
17. The oligonucleotide probe of embodiment 1, having the sequence of SEQ ID NO: 23.
18. A kit comprising the oligonucleotide probe of embodiment 4 and the oligonucleotide probe of embodiment 3 or the oligonucleotide probe of embodiment 2.
19. The kit of embodiment 18, comprising the oligonucleotide probe of embodiment 4, the oligonucleotide probe of embodiment 3 and the oligonucleotide probe of embodiment 2.
20. The kit of embodiment 18 or 19, further comprising a forward primer for the cDNA sequence of the sequence of the N3 gene of the 2019-nCoV, or a fragment thereof, and a reverse primer for the cDNA sequence of the sequence of the N3 gene of the 2019-nCoV, or a fragment thereof.
21. The kit of embodiment 20, wherein the forward primer comprises or consists of the sequence of SEQ ID NO: 7, and the reverse primer comprises or consists of the sequence of SEQ ID NO: 8.
22. The kit of any one of embodiments 18-21, further comprising a forward primer for the cDNA sequence of the sequence of the N2 gene of the 2019-nCoV, or a fragment thereof, and a reverse primer for the cDNA sequence of the sequence of the N2 gene of the 2019-nCoV, or a fragment thereof.
23. The kit of embodiment 22, wherein the forward primer comprises or consists of the sequence of SEQ ID NO: 9, and the reverse primer comprises or consists of the sequence of SEQ ID NO: 10.
24. The kit of any one of embodiments 18-23, further comprising a forward primer for the cDNA sequence of the sequence of the N1 gene of the 2019-nCoV, or a fragment thereof, and a reverse primer for the cDNA sequence of the sequence of the N1 gene of the 2019-nCoV, or a fragment thereof.
25. The kit of embodiment 24, wherein the forward primer comprises or consists of the sequence of SEQ ID NO: 11, and the reverse primer comprises or consists of the sequence of SEQ ID NO: 12.
26. A method of detecting a 2019-nCoV in a sample, comprising:
   (a) providing a sample suspected to contain a 2019-nCoV; and
   (b) subjecting RNA from the sample to a reverse transcription-polymerase chain reaction (RT-PCR) in the presence of an oligonucleotide probe of embodiment 1; and
   (c) detecting fluorescence from the fluorophore of the oligonucleotide probe,
   wherein the presence of fluorescence from the fluorophore of the oligonucleotide probe indicates the sample contains the 2019-nCoV.
27. A method of detecting a 2019-nCoV in a sample, comprising:
   (a) providing a sample suspected to contain a 2019-nCoV;
   (b) subjecting RNA from the sample to a first RT-PCR in the presence of an oligonucleotide probe of embodiment 4, and detecting fluorescence from the fluorophore of the oligonucleotide probe of embodiment 4; and
   (c) subjecting RNA from the sample to a second RT-PCR in the presence of an oligonucleotide probe of embodiment 3 or embodiment 2, and detecting fluorescence from the fluorophore of the oligonucleotide probe of embodiment 3 or embodiment 2.
28. The embodiment of claim 27, wherein the presence of fluorescence from the fluorophore of the oligonucleotide probe of embodiment 4, or the fluorophore of the oligonucleotide probe of embodiment 3 or embodiment 2 indicates the sample contains 2019-nCoV.
29. The method of embodiment 27, wherein:
   (i) the presence of fluorescence from the fluorophore of the oligonucleotide probe of embodiment 4 and the fluorophore of the oligonucleotide probe of embodiment 3 or embodiment 2 indicates the sample contains 2019-nCoV;
   (ii) the absence of fluorescence from the fluorophore of the oligonucleotide probe of embodiment 4 and the fluorophore of the oligonucleotide probe of embodiment 3 or embodiment 2 indicates the sample does not contain 2019-nCoV; and
   (iii) the presence of fluorescence from the fluorophore of the oligonucleotide probe of embodiment 4, but not the fluorophore of the oligonucleotide probe of embodiment 3 or embodiment 2, or the presence of fluorescence from the fluorophore of the oligonucleotide probe of embodiment 3 or embodiment 2, but not the fluorophore of the oligonucleotide probe of embodiment 4 indicates the method is inconclusive with respect to the presence or absence of 2019-nCoV in the sample.
30. A method of detecting a 2019-nCoV in a sample, comprising:

(a) providing a sample suspected to contain a 2019-nCoV;

(b) subjecting RNA from the sample to a first RT-PCR in the presence of an oligonucleotide probe of embodiment 4, and detecting fluorescence from the fluorophore of the oligonucleotide probe of embodiment 4;

(c) subjecting RNA from the sample to a second RT-PCR in the presence of an oligonucleotide probe of embodiment 3, and detecting fluorescence from the fluorophore of the oligonucleotide probe of embodiment 3; and

(d) subjecting RNA from the sample to a third RT-PCR in the presence of an oligonucleotide probe of embodiment 2, and detecting fluorescence from the fluorophore of the oligonucleotide probe of embodiment 2.
31. The method of embodiment 30, wherein the presence of fluorescence from the fluorophore of any one (e.g., one, two or three) of the oligonucleotide probe of embodiment 4, the oligonucleotide probe of embodiment 3 and the oligonucleotide probe of embodiment 2 indicates the sample contains the 2019-nCoV.
32. A method of detecting a 2019-nCoV in a sample, comprising:
   (a) providing a sample suspected to contain a 2019-nCoV;
   (b) subjecting RNA from the sample to RT-PCR in the presence of an oligonucleotide probe of claim 4 and an oligonucleotide probe of embodiment 3 or embodiment 2, wherein the fluorophore of the oligonucleotide probe of embodiment 4 and the fluorophore of the oligonucleotide probe of embodiment 3 or embodiment 2 are independently detectable; and
   (c) detecting fluorescence from the fluorophore of the oligonucleotide probe of embodiment 4 and the fluorophore of the oligonucleotide probe of embodiment 3 or embodiment 2.
33. The method of embodiment 32, wherein the presence of fluorescence from the fluorophore of the oligonucleotide probe of embodiment 4 or the fluorophore of the oligonucleotide probe of embodiment 3 or embodiment 2 indicates the sample contains the 2019-nCoV.
34. The method of embodiment 33, wherein:
   (i) the presence of fluorescence from the fluorophore of the oligonucleotide probe of embodiment 4 and the fluorophore of the oligonucleotide probe of embodiment 3 or embodiment 2 indicates the sample contains the 2019-nCoV;
   (ii) the absence of fluorescence from the fluorophore of the oligonucleotide probe of embodiment 4 and the fluorophore of the oligonucleotide probe of embodiment 3 or embodiment 2 indicates the sample does not contain the 2019-nCoV; and
   (iii) the presence of fluorescence from the fluorophore of the oligonucleotide probe of embodiment 4, but not the oligonucleotide probe of embodiment 3 or embodiment 2, or the presence of fluorescence from the fluorophore of the oligonucleotide of embodiment 3 or embodiment 2, but not the fluorophore of the oligonucleotide probe of embodiment 4 indicates the method is inconclusive with respect to the presence or absence of the 2019-nCoV in the sample.
35. A method of detecting a 2019-nCoV in a sample, comprising:
   (a) providing a sample suspected to contain a 2019-nCoV;
   (b) subjecting RNA from the sample to RT-PCR in the presence of an oligonucleotide probe of embodiment 4, an oligonucleotide probe of embodiment 3 and an oligonucleotide probe of embodiment 2, wherein the fluorophore of the oligonucleotide probe of embodiment 4, the fluorophore of the oligonucleotide probe of embodiment 3 and the fluorophore of the oligonucleotide probe of embodiment 2 are independently detectable; and
   (c) detecting fluorescence from the fluorophore of the oligonucleotide probe of embodiment 4, the fluorophore of the oligonucleotide probe of embodiment 3 and the fluorophore of the oligonucleotide probe of embodiment 2.
36. The method of embodiment 35, wherein the presence of fluorescence from the fluorophore of any one (e.g., one, two or three) of the oligonucleotide probe of embodiment 4, the oligonucleotide probe of embodiment 3 and the oligonucleotide probe of embodiment 2 indicates the sample contains the 2019-nCoV.
37. The method of any one of embodiments 26-36, wherein the sample is from a human.
38. The method of any one of embodiments 26-37, wherein the sample is a nasopharyngeal, oropharyngeal, sputum or stool sample.
39. The method of any one of embodiments 26-38, wherein the method is a method of diagnosing COVID-19 in a subject by detecting a 2019-nCoV in a sample from the subject.

### EXEMPLIFICATION

Instant Quencher-4 (IQ-4, available from ChemGenes Corporation) was benchmarked against BHQ-1 and BHQ-2 in three experiments. In Experiment 1, triplicate 20 μl reactions targeting the housekeeping gene, GAPDH, were prepared for both positive and negative cDNA and three different probes (based on IQ-4, BHQ-1 and BHQ-2). In Experiment 2, five replicate 20 μL reactions for three of the positive control dilutions (400,000; 50,000; and 6,250 copies) as well as a negative human cDNA control were prepared using the three different probes (IQ-4, BHQ-1, BHQ-2) for each of the primer pairs (N1, N2, and N3). In Experiment 3, a sensitivity study was performed using three replicate 20 μL reactions for five dilutions of the 2019-nCoV_N_Positive Control (6,250; 781; 98; 12; and 1 copies).

All probes featured the fluorescein (FAM) fluorophore at the 5′ end of the probe, and one of the three different quenchers, IQ-4, BHQ-1 or BHQ-2, at the 3′ end of the probe. The LOD was tested starting from 400,000 copies to 1 copy of the cDNA molecule using 16-fold dilution series.

### Experimental Design

Reverse transcription of 500 ng of Total Human RNA Control (ThermoFisher, Catalog No. 4307281) was performed by mixing 4 μl of LunaScript RT SuperMix (5×; NEB, Catalog No. E3010), 10 μl of Total Human RNA Control (50 ng/μ1) and 6 μl DEPC water. For No-RT Control Mix (NEB, Catalog No. E3010) reactions, 4 μl of No-RT Control Mix (5×), 10 μl of Total Human RNA Control (50 ng/μ1) and 6 μl DEPC water were combined and mixed briefly. The reactions were incubated in a thermocycler using the following program:

| Cycle Step | Temperature | Time | Cycles |
|---|---|---|---|
| Primer Annealing | 25° C. | 2 minutes | 1 |
| cDNA Synthesis | 55° C. | 10 minutes | 1 |
| Heat Inactivation | 95° C. | 1 minute | 1 |

The resulting cDNA was stored at −20° C.

Triplicate 20 μL reactions targeting the GAPDH gene were prepared for both positive and negative cDNA and the three different probes, IQ-4, BHQ-1 and BHQ-2, for a total of 18 reactions. Each reaction was prepared by mixing 10 μl Luna Universal Probe qPCR Master Mix, 0.8 μl forward primer (10 μM), 0.8 μl reverse primer (10 μM), 0.4 μl probe (10 μM), 2 μl cDNA and 6 μl DEPC water. qPCR was performed in a Roche LightCycler® 480 system (Roche Life Science) set up to detect FAM (Ex/Em 495/520 nm) using the following program:

| Cycle Step | Temperature | Time | Cycles |
|---|---|---|---|
| Initial Denaturation | 95° C. | 60 seconds | 1 |
| Denaturation | 95° C. | 15 seconds | 40 |
| Extension | 60° C. | 30 seconds | |

Glyceraldehyde 3-phosphate dehydrogenase (GAPDH) is a well-known housekeeping gene with diverse functions in cellular homeostasis and glycolysis. This primarily cytoplasmic protein is an essential metabolic regulator and has been shown to be involved in a variety of cellular processes like DNA repair, membrane fusion, and cell death. Cytoplasmic GAPDH exists as a tetramer and normally mediates the formation of ATP and NADH during glycolysis. Under oxidative stress, GAPDH can be post-translationally modified to regulate cell metabolism. Additionally, GAPDH has been shown to interact with the cytoskeleton to influence microtubule and actin polymerization. The following probe and primer sequences were used for the experiments involving the GAPDH gene:

| | Sequence | |
|---|---|---|
| SEQ ID NO | Description | Sequence |
| SEQ ID NO: 3 | Forward | CCCATGTTCGTCATGGGTGT |
| | Primer | |
| SEQ ID NO: 13 | Reverse | GGTCATGAGTCCTTCCACGATA |
| | Primer | |
| SEQ ID NO: 14 | Probe-1 | 6-FAM-CTGCACCACCAACTGC |
| | | TTAGCACCC-IQ-4 |
| SEQ ID NO: 15 | Probe-2 | 6-FAM-CTGCACCACCAACTGC |
| | | TTAGCACCC-BHQ1 |
| SEQ ID NO: 16 | Probe-3 | 6-FAM-CTGCACCACCAACTGC |
| | | TTAGCACCC-BHQ2 |

The following probe and primer sequences can be used to detect BRCA1 using qPCR:

| | Sequence | |
|---|---|---|
| SEQ ID NO | Description | Sequence |
| SEQ ID NO: 26 | Forward | ACAGCTGTGTGGTGCTTCTGTG |
| | Primer | |
| SEQ ID NO: 27 | Reverse | CATTGTCCTCTGTCCAGGCATC |
| | Primer | |
| SEQ ID NO: 1 | Probe-1 | 6-FAM-CATCATTCACCCTTGG |
| | | CACAGGTGT-IQ-4 |
| SEQ ID NO: 2 | Probe-2 | 6-FAM-CATCATTCACCCTTGG |
| | | CACAGGTGT-BHQ2 |

The 2019-nCov_N_Positive Control was obtained from IDT (Catalog No. 10006625), and contains approximately 200,000 copies of a plasmid with the complete 2019-nCoV nucleocapsid gene per microliter. Serial 8-fold dilutions were made from the 2019-nCoV_N_Positive Control, which was estimated to correspond to three threshold cycle (Ct) value differences (Table 3). The cDNA reverse transcribed from Total Human RNA Control and 5× LunaScript RT Super Mix was used as a negative control. A negative cDNA control was reverse transcribed using Total Human RNA Control, and used as an extraction control.

TABLE 3
Serial dilutions made from 2019-nCoV_N_Positive Control and the
corresponding number of copies and estimated Ct value for qPCR.

| Dilution | Total number of copies per reaction | Estimated Ct |
|---|---|---|
| 1 | 400,000 | 23 |
| 1:8 | 50,000 | 26 |
| 1:64 | 6,250 | 29 |
| 1:512 | 781 | 32 |
| 1:4096 | 98 | 35 |
| 1:32,768 | 12 | 38 |
| 1:262,144 | 1 | 41 |

Five replicate 20-4, reactions for three of the positive control dilutions (400,000; 50,000; and 6,250 copies), as well as the negative human cDNA control were prepared using the three different probes (IQ-4, BHQ-1, BHQ-2) for a total of 60 reactions per run for each of the primer pairs (N1, N2, and N3).

A sensitivity study was performed using three replicate 20-4, reactions for five dilutions of the 2019-nCoV_N_Positive Control (6,250; 781; 98; 12; and 1 copies).

The primers and probes utilized in each of the experiments were synthesized simultaneously in-house at ChemGenes Corporation under the same conditions. The Luna Universal Probe qPCR Master Mix (NEB, Catalog No. M3004) was used for all qPCR reactions, and all qPCR reactions were performed on the LightCycler® 480 II instrument (Roche, Catalog No. 05015278001) with the same settings. Initial denaturation was performed at 95° C. for 1 minute followed by 40 cycles of denaturation at 95° C. for 15 seconds and extension at 60° C. for 30 seconds. The detection format was set to detect FAM (Ex/Em 483 nm/533 nm). For the sensitivity study, the number of PCR cycles was increased to 48.

The following probe and primer sequences were used for the experiments involving 2019-nCoV:

| | | Sequence | |
|---|---|---|---|
| Gene | SEQ ID NO | Description | Sequence |
| 2019- | SEQ ID NO: 7 | Forward-Primer | GACCCCAAAATCAGCGAAAT |
| nCoV_N1 | SEQ ID NO: 8 | Reverse Primer | TCTGGTTACTGCCAGTTGAATCTG |
| | SEQ ID NO: 17 | Probe-1 | 6-FAM-ACCCCGCATTACGTTTGGTGGACC-IQ-4 |
| | SEQ ID NO: 18 | Probe-2 | 6-FAM-ACCCCGCATTACGTTTGGTGGACC-BHQ1 |
| | SEQ ID NO: 19 | Probe-3 | 6-FAM-ACCCCGCATTACGTTTGGTGGACC-BHQ2 |
| 2019- | SEQ ID NO: 9 | F-Primer | TTACAAACATTGGCCGCAAA |
| nCoV_N2 | SEQ ID NO: 10 | R-Primer | GCGCGACATTCCGAAGAA |
| | SEQ ID NO: 20 | Probe-1 | 6-FAM-ACAATTTGCCCCCAGCGCTTCAG-IQ-4 |
| | SEQ ID NO: 21 | Probe-2 | 6-FAM-ACAATTTGCCCCCAGCGCTTCAG-BHQ1 |
| | SEQ ID NO: 22 | Probe-3 | 6-FAM-ACAATTTGCCCCCAGCGCTTCAG-BHQ2 |
| 2019- | SEQ ID NO: 11 | F-Primer | GGGAGCCTTGAATACACCAAAA |
| nCoV_N3 | SEQ ID NO: 12 | R-Primer | TGTAGCACGATTGCAGCATTG |
| | SEQ ID NO: 23 | Probe-1 | 6-FAM-AYCACATTGGCACCCGCAATCCTG-IQ-4 |
| | SEQ ID NO: 24 | Probe-2 | 6-FAM-AYCACATTGGCACCCGCAATCCTG-BHQ1 |
| | SEQ ID NO: 24 | Probe-3 | 6-FAM-AYCACATTGGCACCCGCAATCCTG-BHQ2 |

### Results and Discussion

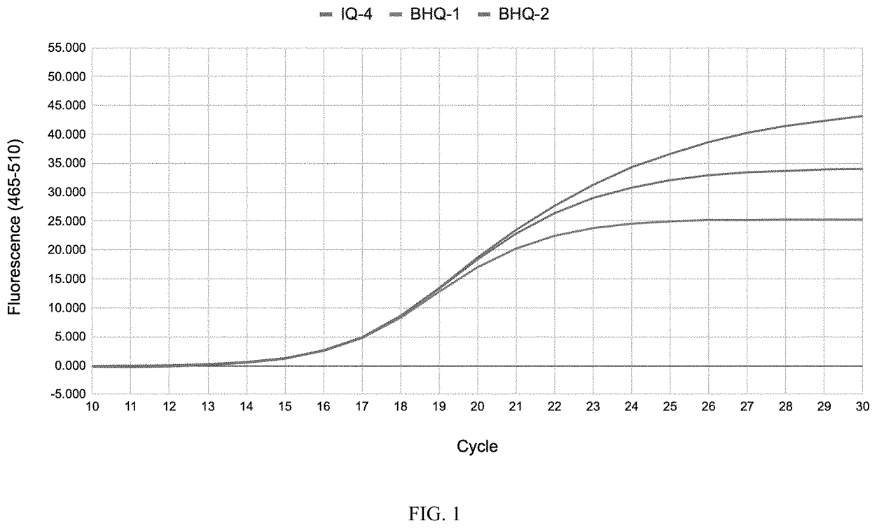

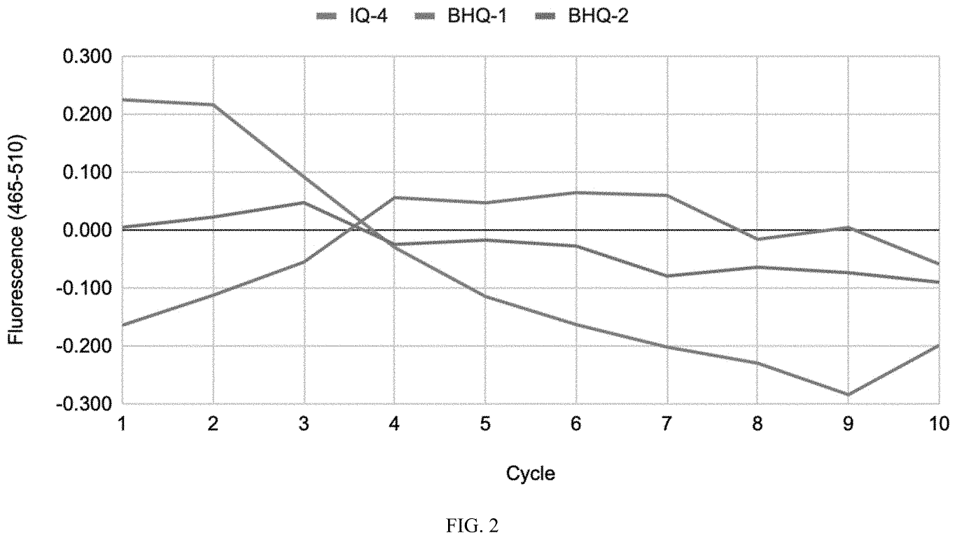

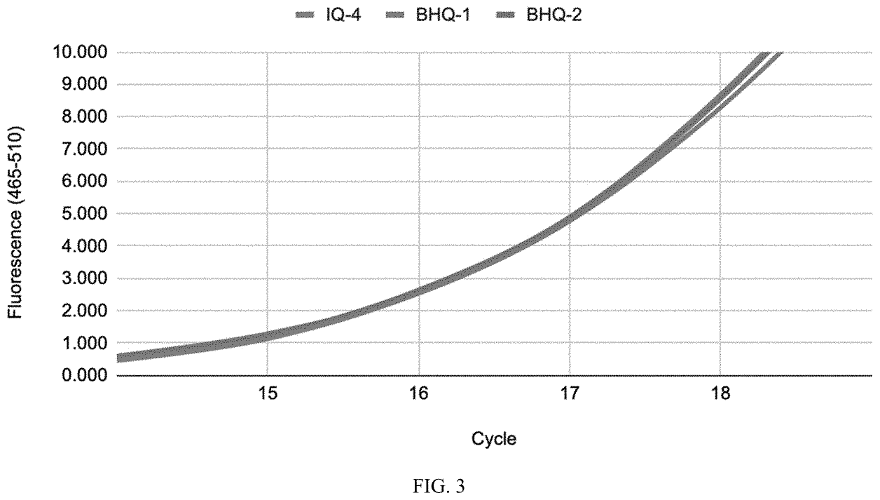

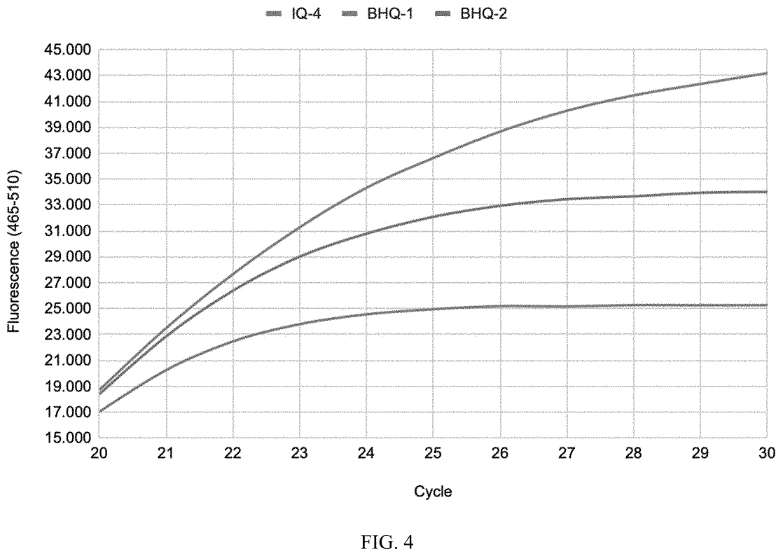

To compare the quenching efficiency of IQ-4 with BHQ-1 and BHQ-2, three parameters were observed when targeting the GAPDH gene: (1) Mean Ct value; (2) background noise level; and (3) maximum fluorescence. Table 4 reports the mean Ct value for the three probes when targeting a high-expression gene, GAPDH. FIGS. 1-4 show the fluorescence data obtained from this experiment.

TABLE 4

| Quencher | Mean Ct |
|---|---|
| IQ4 | 15.35 |
| BHQ1 | 15.41 |
| BHQ2 | 15.32 |

To compare the quenching efficiency of IQ-4 with BHQ-1 and BHQ-2 when targeting the three target genes of 2019-nCoV (N1, N2, and N3), the following parameters were observed: (1) mean Ct value; and (2) background noise level. Average values were calculated using the corrected mean after removing outliers. Table 5 reports the average Ct values for each of the three targeted genes (N1, N2, N3) and dilutions (400,000; 50,000; and 6,250 copies) of the 2019_nCov positive control and each of the three quenchers (IQ-4, BHQ-1, BHQ-2). An eight-fold dilution corresponds to a difference in approximately three cycles. Table 6 reports The standard deviation of each quencher from the average Ct value.

TABLE 5

| | | 400,000 | 50,000 | 6,250 | Human |
|---|---|---|---|---|---|
| | | copies | copies | copies | cDNA |
| 2019-nCov N1 | | | | | |
| Quencher | IQ-4 | 23.38 | 26.796 | 29.642 | NEG |
| | BHQ1 | 22.998 | 26.352 | 29.234 | NEG |
| | BHQ2 | 23.442 | 26.648 | 29.606 | NEG |
| 2019-nCov N2 | | | | | |
| Quencher | IQ-4 | 23.35 | 26.67 | 29.706 | NEG |
| | BHQ1 | 23.13 | 26.082 | 29.144 | NEG |
| | BHQ2 | 23.75 | 26.584 | 29.654 | NEG |
| 2019-nCov N3 | | | | | |
| Quencher | IQ-4 | 24.424 | 27.192 | 30.354 | NEG |
| | BHQ1 | 23.526 | 26.618 | 29.672 | NEG |
| | BHQ2 | 23.834 | 26.692 | 29.852 | NEG |

TABLE 6

| | | 400,000 copies | 50,000 copies | 6,250 copies |
|---|---|---|---|---|
| 2019-nCov N1 | | | | |
| Quencher | IQ-4 | 0.203 | 0.209 | 0.199 |
| | BHQ1 | 0.240 | 0.222 | 0.226 |
| | BHQ2 | 0.214 | 0.186 | 0.193 |
| 2019-nCov N2 | | | | |
| Quencher | IQ-4 | 0.258 | 0.283 | 0.273 |
| | BHQ1 | 0.292 | 0.317 | 0.310 |
| | BHQ2 | 0.308 | 0.268 | 0.265 |
| 2019-nCov N3 | | | | |
| Quencher | IQ-4 | 0.448 | 0.312 | 0.350 |
| | BHQ1 | 0.423 | 0.277 | 0.322 |
| | BHQ2 | 0.376 | 0.265 | 0.294 |

A sensitivity study was conducted to test the limit of detection of the three probes with between a range of 6,250 copies and 1 copy of the 2019-nCoV_N_Positive Control plasmid. Table 7 reports the average Ct values for each of the three targeted genes (N1, N2, N3) and dilutions (number of copies) of the 2019 nCov positive control and each of the three quenchers (IQ-4, BHQ-1, BHQ-2). An eight-fold dilution corresponds to a difference in approximately three cycles.

TABLE 7

| | Number of copies | 6,250 | 781 | 98 | 12 | 1 |
|---|---|---|---|---|---|---|
| 2019-nCov N1 | | | | | | |
| Quencher | IQ-4 | 31.1 | 33.4 | 35.3 | 38.0 | n/a |
| | BHQ1 | 31.9 | 33.5 | 36.6 | 37.5 | n/a |
| | BHQ2 | 32.2 | 34.4 | 36.7 | n/a | n/a |
| 2019-nCov N2 | | | | | | |
| Quencher | IQ-4 | 32.9 | 34.9 | 37.1 | 38.4 | n/a |
| | BHQ1 | 32.6 | 34.4 | 35.8 | n/a | n/a |
| | BHQ2 | 32.8 | 34.4 | 36.1 | n/a | n/a |
| 2019-nCov N3 | | | | | | |
| Quencher | IQ-4 | 33.0 | 34.9 | 37.5 | 39.0 | n/a |
| | BHQ1 | 32.3 | 34.8 | 36.1 | n/a | n/a |
| | BHQ2 | 32.9 | 35.0 | 36.5 | n/a | n/a |

### CONCLUSIONS

When benchmarked against the BHQ-1 and BHQ-2 quenchers, IQ-4 has been found to have similar performance in Ct value and quenching efficiency to the current industry-leading quenchers and CDC-approved BHQ-1. However, the background fluorescence of IQ-4 was found to be lower than that of BHQ-1 and BHQ-2.

The sensitivity experiment in this study indicated that the IQ-4 quencher had the highest sensitivity of the three quenchers tested, and was able to detect down to 12 copies of the 2019-nCoV plasmid for all three genes. In contrast, BHQ-1 was able to detect 12 copies of the N1 gene, but not the N2 and N3 genes, and BHQ-2 was unable to detect 12 copies of any of the three genes.

These results are critical from a public health standpoint. Currently, the diagnostic qPCR test only includes two of the 2019-nCoV genes (N1 and N2) due to the low sensitivity of the third gene (N3). If one of the two genes is not detected with the test, the results are recorded as “undetermined” and the patient must be retested, consuming more supplies and increasing the queue of cases. Based on the results of this study, it can be hypothesized that most of the undetermined tests using BHQ-1 are from negative results for gene N2.

By creating a diagnostic test that includes all three genes (N1, N2, and N3), the probability of detecting the presence of 2019-nCoV can be increased, and difficulties in assigning or predicting results with just two genes avoided.

This study has demonstrated that the IQ-4 quencher provides benefits over BHQ-1 and BHQ-2, which are currently limited in terms of availability. The IQ-4 quencher represents not only an excellent alternative to BHQ-1 and BHQ-2, and offers higher sensitivity that could improve detection of the N2 gene and allow testing of all three of the 2019-nCoV genes, increasing the number of reliable test results and closing the gap of undetermined cases.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A panel of oligonucleotide probes for detecting a 2019 novel coronavirus (2019-nCoV), comprising a first oligonucleotide probe, a second oligonucleotide probe and a third oligonucleotide probe, wherein:
the first oligonucleotide probe comprises a probe sequence consisting of the sequence of SEQ ID NO: 4, or a sequence having at least about 90% identity to the sequence of SEQ ID NO: 4;
the second oligonucleotide probe comprises a probe sequence consisting of the sequence of SEQ ID NO: 5, or a sequence having at least about 90% identity to the sequence of SEQ ID NO: 5; and
the third oligonucleotide probe comprises a probe sequence consisting of the sequence of SEQ ID NO: 6, or a sequence having at least about 90% identity to the sequence of SEQ ID NO: 6,
wherein each probe sequence is independently modified at its 5′ terminus with a fluorescein fluorophore having an emission maximum of from about 500 nm to about 710 nm and at its 3′ terminus with a moiety of the following structural formula: 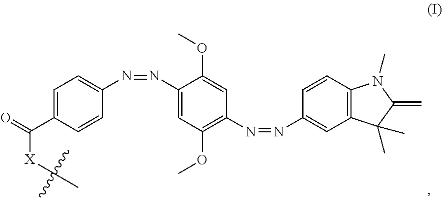
wherein: [symbol]
indicates the point of attachment of the moiety to the 3′ terminus of the probe sequence; and
X is a linker.

2-5. (canceled)

6. The panel of claim 1, wherein:
the first oligonucleotide probe comprises a probe sequence consisting of the sequence of SEQ ID NO: 4;
the second oligonucleotide probe comprises a probe sequence consisting of the sequence of SEQ ID NO: 5; and
the third oligonucleotide probe comprises a probe sequence consisting of the sequence of SEQ ID NO: 6.

7-10. (canceled)

11. The panel of claim 1, wherein the fluorescein fluorophore is 6-carboxyfluorescein, tetrachlorofluorescein, or hexachlorofluorescein.

12. The panel of claim 11, wherein the fluorescein fluorophore is 6-carboxyfluorescein.

13. The panel of claim 1, wherein X is (C₁-C₂₅)alkylene, (C₁-C₂₅)alkenylene, (C₁-C₂₅)alkynylene, (C₁-C₂₅)heteroalkylene, (C₁-C₂₅)heteroalkenylene or (C₁-C₂₅)heteroalkynylene.

14. The panel of claim 13, wherein X is 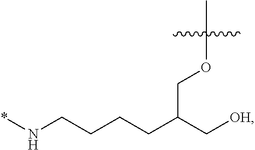 [SMILES: *NCCCCC(CO)CO*]
wherein * indicates the point of attachment of X to the carbonyl of structural formula I.

15. The panel of claim 1, wherein the first oligonucleotide probe has the sequence of SEQ ID NO: 17.

16. The panel of claim 1, wherein the second oligonucleotide probe has the sequence of SEQ ID NO: 20.

17. The panel of claim 1, wherein the third oligonucleotide probe has the sequence of SEQ ID NO: 23.

18. A kit for detecting a 2019-nCoV, comprising:
a panel of claim 1;
a forward primer and a reverse primer for a target of the first oligonucleotide probe;
a forward primer and a reverse primer for a target of the second oligonucleotide probe; and
a forward primer and a reverse primer for a target of the third oligonucleotide probe.

19-24. (canceled)

25. A method of detecting a 2019 novel coronavirus (2019-nCoV) in a sample, comprising:
(a) providing a sample suspected to contain a 2019-nCoV;
(b) subjecting RNA from the sample to a first RT-PCR in the presence of a first oligonucleotide probe comprising a probe sequence consisting of the sequence of SEQ ID NO: 6, or a sequence having at least about 90% identity to the sequence of SEQ ID NO: 6, modified at its 5′ terminus with a fluorescein fluorophore having an emission maximum of from about 500 nm to about 710 nm and at its 3′ terminus with a moiety of structural formula (I), and detecting fluorescence from the fluorescein fluorophore of the first oligonucleotide probe;
(c) subjecting RNA from the sample to a second RT-PCR in the presence of a second oligonucleotide probe comprising a probe sequence consisting of the sequence of SEQ ID NO: 5, or a sequence having at least about 90% identity to the sequence of SEQ ID NO: 5, modified at its 5′ terminus with a fluorescein fluorophore having an emission maximum of from about 500 nm to about 710 nm and at its 3′ terminus with a moiety of structural formula (I), and detecting fluorescence from the fluorescein fluorophore of the second oligonucleotide probe; and
(d) subjecting RNA from the sample to a third RT-PCR in the presence of a third oligonucleotide probe comprising a probe sequence consisting of the sequence of SEQ ID NO: 4, or a sequence having at least about 90% identity to the sequence of SEQ ID NO: 4, modified at its 5′ terminus with a fluorescein fluorophore having an emission maximum of from about 500 nm to about 710 nm and at its 3′ terminus with a moiety of structural formula (I), and detecting fluorescence from the fluorescein fluorophore of the third oligonucleotide probe,
wherein:
each probe sequence is independently modified at its 5′ terminus with a fluorescein fluorophore having an emission maximum of from about 500 nm to about 710 nm and at its 3′ terminus with a moiety of the following structural formula: 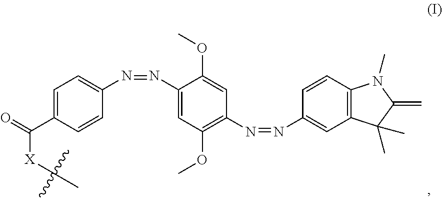
wherein: [symbol]
indicates the point of attachment of the moiety to the 3′ terminus of the probe sequence; and
X is a linker.

26. (canceled)

27. A method of detecting a 2019 novel coronavirus (2019-nCoV) in a sample, comprising:
(a) providing a sample suspected to contain a 2019-nCoV;
(b) subjecting RNA from the sample to RT-PCR in the presence of first, second and third oligonucleotide probes, wherein:
the first oligonucleotide probe comprises a probe sequence consisting of the sequence of SEQ ID NO: 6, or a sequence having at least about 90% identity to the sequence of SEQ ID NO: 6;
the second oligonucleotide probe comprises a probe sequence consisting of the sequence of SEQ ID NO: 5, or a sequence having at least about 90% identity to the sequence of SEQ ID NO: 5; and
the third oligonucleotide probe comprises a probe sequence consisting of the sequence of SEQ ID NO: 4, or a sequence having at least about 90% identity to the sequence of SEQ ID NO: 4, wherein:
each probe sequence is independently modified at its 5′ terminus with a fluorescein fluorophore having an emission maximum of from about 500 nm to about 710 nm and at its 3′ terminus with a moiety of the following structural formula: 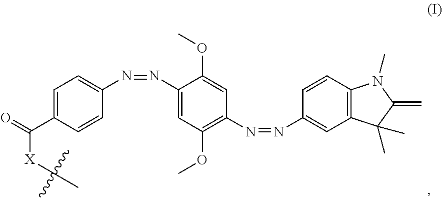
wherein: [symbol]
indicates the point of attachment of the moiety to the 3′ terminus of the probe sequence; and
X is a linker; and
the fluorescein fluorophore of the first oligonucleotide probe, the fluorescein fluorophore of the second oligonucleotide probe and the fluorescein fluorophore of the third oligonucleotide probe are independently detectable; and
(c) detecting fluorescence from the fluorescein fluorophore of the first oligonucleotide probe, the fluorescein fluorophore of the second oligonucleotide probe and the fluorescein fluorophore of the third oligonucleotide probe.

28. The method of claim 27, wherein the sample is from a human.

29. The method of claim 27, wherein the sample is a nasopharyngeal, oropharyngeal, sputum or stool sample.

30. The method of claim 27, wherein the method is a method of diagnosing COVID-19 in a subject by detecting a 2019-nCoV in a sample from the subject.

31. The method of claim 25, wherein each probe sequence is modified at its 5′ terminus with the same fluorescein fluorophore.

32. The method of claim 25, wherein the fluorescein fluorophores of the first oligonucleotide probe, second oligonucleotide probe and third oligonucleotide probe are independently detectable.

33. The method of claim 25, wherein the sample is a from a human.

34. The method of claim 25, wherein the sample is a nasopharyngeal, oropharyngeal, sputum or stool sample.

35. The method of claim 25, wherein the method is a method of diagnosing COVID-19 in a subject by detecting a 2019-nCoV in a sample from the subject.

36. The panel of claim 1, wherein each probe sequence is modified at its 5′ terminus with the same fluorescein fluorophore.

37. The panel of claim 1, wherein the fluorescein fluorophores of the first oligonucleotide probe, second oligonucleotide probe and third oligonucleotide probe are independently detectable.
